(19) United States
(12) Patent Application Publication
(10) Pub. No.: US 2025/0212895 A1
(43) Pub. Date: Jul. 3, 2025

(54) TARGETED CONTROL OF WEEDS AND INVASIVE PLANTS BY DELIVERY OF 2'FANA ANTI-SENSE OLIGONUCLEOTIDES

(71) Applicants: Colorado State University Research Foundation, Fort Collins, CO (US); AUM LifeTech, Inc., Philadelphia, PA (US)

(72) Inventors: Todd Gaines, Fort Collins, CO (US); Veenu Aishwarya, Philadelphia, PA (US); Katie Martin, Fort Collins, CO (US)

(21) Appl. No.: 18/841,641

(22) PCT Filed: Mar. 1, 2023
(86) PCT No.: PCT/US2023/063487

§ 371 Date: Aug. 26, 2024

Related U.S. Application Data
(60) Provisional application No. 63/315,211, filed on Mar. 1, 2022.

(51) Int. Cl. A01N 63/60; A01P 13/02; C12N 5/04

(52) U.S. Cl. CPC A01N 63/60; A01P 13/02; C12N 5/04; C12N 2510/00

(57) ABSTRACT

The disclosure relates to 2′-deoxy-2′-fluoroarabinonucleic acid (FANA) antisense oligonucleotides that can be utilized to control weeds, invasive plants, or other undesired plants.

### CROSS REFERENCE TO RELATED APPLICATIONS

This application claims priority to provisional application U.S. Ser. No. 63/315,211, filed Mar. 1, 2022, which is incorporated herein by reference in its entirety.

### SEQUENCE LISTING XML

The instant application contains a sequence listing, which has been submitted in XML file format by electronic submission and is hereby incorporated by reference in its entirety. The XML file, created on Feb. 28, 2023, is named P14317WO00.xml and is 1,098,971 bytes in size.

### TECHNICAL FIELD

The present disclosure relates to the field of biotechnology. More specifically, the present disclosure relates to compositions and methods for control of weeds, invasive plants, and any undesired plant vegetation.

### BACKGROUND

Weeds cause economic losses in crop production due to competition for light, water, and nutrients. Weed control can be conducted by labor-intensive hand operations, by mechanical means, and in modern agriculture, most often by efficient and effective herbicides. Herbicides are defined as small molecules synthesized to inhibit specific proteins in plants or to interfere with electron transport chains in plants and generate reactive oxygen species. Herbicides reduce the amount of time, labor, and fuel required to control weeds. Cost-effective and safe herbicides have been discovered and commercialized for nearly every crop and landscape market. However, highly effective herbicides exert substantial selection pressure on weed populations for resistance to the herbicides. Any genetic trait conferring higher survival can be selected and enriched in subsequent generations of the weed species. Herbicide resistance has become a critical problem for many cropping production systems. Many problematic weed species including kochia, Palmer amaranth, waterhemp, ryegrass, blackgrass, barnyardgrass, horseweed, redroot pigweed, and common lambsquarters have evolved resistance to the major herbicides used for their control. Thus, there is need in the art for alternative weed control methods.

### SUMMARY

Described herein is the use of 2′-deoxy-2′-fluoroarabinonucleic acid (FANA) antisense oligonucleotides (ASOs), delivered alone or in combination with nanoparticles, to bind mRNA transcripts of essential plant target genes, triggering mRNA cleavage by the enzyme RNAse H that eliminates the bound mRNA transcript and reduces the concentration of the protein encoded by the target mRNA to a level that is lethal to the plant. FANA antisense oligonucleotides can also inhibit translation of mRNAs by binding tightly to the transcript thus preventing ribosomal assembly.

Methods of controlling weeds are provided. The methods comprise providing to the weed a composition comprising an antisense oligonucleotide comprising at least one FANA-modified nucleotide, wherein the antisense oligonucleotide targets a transcript of an essential gene of the weed. Methods of inducing gene silencing in a plant are also provided.

Antisense oligonucleotides comprising at least one FANA-modified nucleotide and capable of targeting a transcript of an essential gene of a plant are provided. In certain embodiments, the antisense oligonucleotides comprise a sequence complementary to at least 18, at least 19, at least 20, at least 21, at least 22, at least 23, at least 24, or at least 25 contiguous nucleotides of a transcript of an essential gene of the plant. Herbicidal compositions and formulations comprising the antisense oligonucleotides are also provided.

Plants and plant cells comprising the antisense oligonucleotide compositions are provided. Plant seeds coated with the antisense oligonucleotide compositions are also provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A is a schematic showing that FANA single-stranded antisense oligonucleotides use RNase H-mediated RNA cleavage. Following cleavage, the fragmented mRNA is further degraded by nucleases, whereas the FANA antisense oligonucleotides are recycled within the cell. FIG. 1B is a schematic showing the RNAi pathway requires the interaction of several enzymes (RISC complex).

FIG. 2A-F shows uptake of a fluorescent FAM-labeled FANA antisense oligonucleotide into plant roots and leaves. FIG. 2A-B shows an untreated control root and FIG. 2C-D shows a treated root. FIG. 2E shows an untreated control leaf and FIG. 2F shows a treated leaf.

FIG. 3A-D shows FANA antisense oligonucleotides caused herbicidal symptoms when applied using syringe injection. Scramble FANA antisense oligonucleotide showed no necrosis (FIG. 3A) while ATPC1 gene target shows significant leaf necrosis under syringe injection (FIG. 3B). No effect was observed when applied as foliar spray for scramble (FIG. 3C) or ATPC1 (FIG. 3D).

FIG. 4A-B shows the lethal effect of a FANA antisense oligonucleotide targeting the HPPD gene (HPPD_SEQ1) in Palmer amaranth when applied as syringe injection to the underside of plant leaves. There was a lack of lethal effect from water only control and from the scramble treatment; some leaf damage in water and scramble treatments due to drought stress that occurred. HPPD1 treatment shows typical HPPD-inhibitor injury. Photographs taken 18 days after treatment.

FIG. 5 shows FANA antisense oligonucleotides reach the nucleus. Protoplasts were isolated from Arabidopsis and treated with a fluorescent FANA antisense oligonucleotide (ASO) or carbon dots with the FANA antisense oligonucleotide (CD-ASO).

FIG. 6 shows carbon dots improve uptake of FANA antisense oligonucleotides. Arabidopsis leaves were infiltrated with a fluorescent FANA antisense oligonucleotide (ASO) or carbon dots with the FANA antisense oligonucleotide (CD-ASO). The carbon dot treated areas showed more cytoplasm uptake of the fluorescent FANA antisense oligonucleotide. Images with FITC filter at 20× magnification shown.

FIG. 7 shows the results of a luciferase assay performed on leaves treated with a fluorescent FANA antisense oligonucleotide (ASO) or carbon dots with the FANA antisense oligonucleotide (CD-ASO). The results indicate that carbon dots may improve FANA antisense oligonucleotide uptake into the plant.

FIG. 8A-B shows a multiple sequence alignment of the ATPC1 sequences for Palmer amaranth (AMAPA, Amaranthus palmeri) (SEQ ID NO: 6) with the crops maize (Zea mays) (SEQ ID NO: 357), soybean (Glycine max) (SEQ ID NO: 419), and wheat (Triticum aestivum) (SEQ ID NO: 481). Five FANA antisense oligonucleotide sequences for ATPC1 in Palmer amaranth (SEQ ID NOs: 551-555) are shown in sense orientation (SEQ ID NOs: 568-572).

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, for example, reference to “a mutation” includes a single mutation, as well as two or more mutations; reference to “a plant” includes one plant, as well as two or more plants; and so forth. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the exact RNA equivalent. A given first polynucleotide sequence, whether DNA or RNA, further defines the sequence of its exact complement (which can be DNA or RNA), a second polynucleotide that hybridizes perfectly to the first polynucleotide by forming Watson-Crick base-pairs. For DNA: DNA duplexes (hybridized strands), base-pairs are adenine: thymine or guanine: cytosine; for DNA: RNA duplexes, base-pairs are adenine: uracil or guanine: cytosine.

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. An appropriate “effective amount” can be determined by the skilled artisan via routine experimentation.

As used herein, the term “essential gene” refers to a gene of an organism that is essential for its survival or reproduction.

As used herein, the term “foliage” is intended to mean all parts and organs of plants above the ground. Non-limiting examples include leaves, stalks, stems, flowers, fruits, etc.

As used herein, the term “foliar application”, “foliarly applied”, and variations thereof, is intended to include application to the foliage or above ground portions of the plant, (e.g., the leaves of the plant).

As used herein, the terms “include,” “includes,” and “including” are to be construed as at least having the features to which they refer while not excluding any additional unspecified features.

As used herein, the term “modified antisense oligonucleotide” refers to antisense oligonucleotides (ASOs) containing a modified sugar. Antisense oligonucleotides are single stranded oligonucleotides that recognize nucleic acid sequences via Watson-Crick base pairing and cause pre- or post-transcriptional gene silencing. The antisense oligonucleotide binds to its target mRNA, and forms a duplex that is recognized by RNase H, which in turn induces the cleavage of the mRNA, the steric blocking of translation machinery, or the prevention of necessary RNA interactions.

A “naturally occurring nucleotide” or “unmodified nucleotide” contains normally occurring sugars (D-ribose or D-2-deoxyribose) and a phosphodiester backbone that is readily degraded by nucleases. As used herein, an unmodified nucleotide is also referred to as a deoxyribonucleotide.

The terms “plants” and “vegetation” include, but are not limited to, germinant seeds, emerging seedlings, plants emerging from vegetative propagules, and established vegetation.

The term “weed” refers to all those plants which grow in locations where they are undesired. The terms “undesirable vegetation”, “harmful plants”, “unwanted plants”, “weeds” and “weed species”, as used herein, are synonyms. The weeds of the present disclosure can also include, for example, crop plants that are growing in an undesired location. For example, a volunteer maize plant that is in a field that predominantly comprises soybean plants can be considered a weed, if the maize plant is undesired in the field of soybean plants.

### FANA Antisense Oligonucleotides

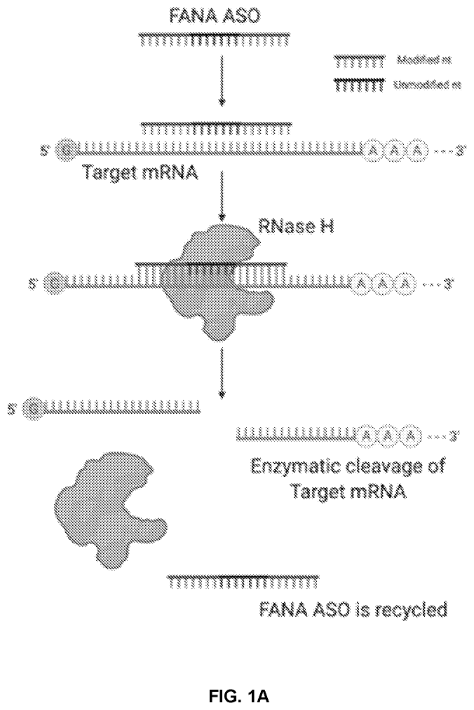

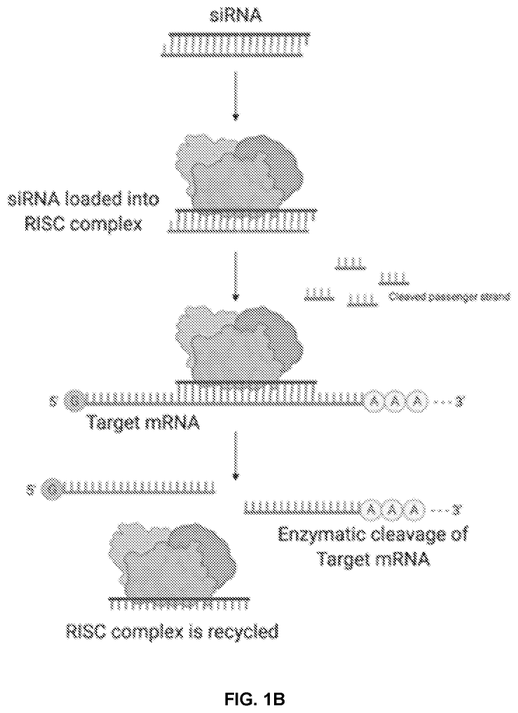

FANA antisense oligonucleotides are modified synthetic single-stranded nucleic acid analogs that can modulate gene expression by enzymatic degradation or steric blocking of an RNA target. FANA antisense oligonucleotides can recognize and bind to specific RNA forms, including mRNA, through complementary base pairing. Contrary to the RNAi pathway that requires the interaction of several enzymes (RISC complex), FANA single-stranded antisense oligonucleotides use RNase H-mediated RNA cleavage (FIG. 1). Ribonuclease H (RNase H), an endogenous enzyme present in both prokaryotes and eukaryotes, recognizes the FANA/mRNA duplex and cleaves the RNA within the hybrid. Following cleavage, the fragmented mRNA is further degraded by nucleases, whereas the FANA antisense oligonucleotides are recycled within the cell; the degradation of multiple mRNA copies by a single FANA antisense oligonucleotide increases the silencing efficiency and lowers the dose required. Furthermore, unlike siRNAs that are only processed in the cytoplasm, FANA antisense oligonucleotides can shuttle into the nucleus of eukaryotic cells and silence nuclear RNA. FANA antisense oligonucleotides that do not induce RNase H cleavage can inhibit translation of mRNAs, by binding tightly to the transcript thus preventing ribosomal assembly. FANA antisense oligonucleotides have substantial advantages over a dsRNA system, including higher stability in storage, stability at room temperature, and longer stability and resistance to degradation within the cell.

The chemistry and construction of FANA antisense oligonucleotides has been described elsewhere in detail (see, e.g., U.S. Pat. Nos. 8,278,103 and 9,902,953, each of which is specifically incorporated herein in their entirety by reference). The FANA antisense oligonucleotides and methods of using them disclosed herein contemplate any FANA chemistries known in the art. In some embodiments, a FANA antisense oligonucleotide comprises an internucleoside linkage comprising a phosphate, thereby being an oligonucleotide. In some embodiments, the sugar-modified nucleosides and/or 2′-deoxynucleosides comprise a phosphate, thereby being sugar-modified nucleotides and/or 2′-deoxynucleotides. In some embodiments, a FANA antisense oligonucleotide comprises an internucleoside linkage comprising a phosphorothioate. In some embodiments, the internucleoside linkage is selected from phosphorothioate, phosphorodithioate, methylphosphorothioate, Rp-phosphorothioate, Sp-phosphorothioate. In some embodiments, a FANA antisense oligonucleotide comprises one or more internucleotide linkages selected from (a) phosphodiester; (b) phosphotriester; (c) phosphorothioate; (d) phosphorodithioate; (e) Rp-phosphorothioate; (f) Sp-phosphorothioate; (g) boranophosphate; (h) methylene (methylimino) (3′CH₂—N(CH₃)—O5′); (i) 3′-thioformacetal (3'S—CH₂—O5′); (j) amide (3′CH₂—C(O)NH-5′); (k) methylphosphonate; (l) phosphoramidate (3′-OP(O₂)—N5′); and (m) any combination of (a) to (l).

In some embodiments, FANA antisense oligonucleotides comprising alternating segments or units of sugar-modified nucleotides (e.g., arabinonucleotide analogues [e.g., FANA]) and 2′-deoxyribonucleotides (DNA) are utilized. In some embodiments, a FANA antisense oligonucleotide disclosed herein comprises at least 2 of each of sugar-modified nucleotide and 2′-deoxynucleotide segments, thereby having at least 4 alternating segments overall. Each alternating segment or unit may independently contain 1 or a plurality of nucleotides. In some embodiments, each alternating segment or unit may independently contain 1 or 2 nucleotides. In some embodiments, the segments each comprise 1 nucleotide. In some embodiments, the segments each comprise 2 nucleotides. In some embodiments, the plurality of nucleotides may consist of 2, 3, 4, 5, or 6 nucleotides. A FANA antisense oligonucleotide can contain an odd or even number of alternating segments or units and may commence and/or terminate with a segment containing sugar-modified nucleotide residues or DNA residues. Thus, a FANA antisense oligonucleotide can be represented as follows:
A₁-D₁-A₂-D₂-A₃-D₃ . . . A_{z}-D_{z}
   where each of A₁, A₂, etc. represents a unit of one or more (e.g., 1 or 2) sugar-modified nucleotide residues (e.g., FANA) and each of D₁, D₂, etc. represents a unit of one or more (e.g., 1 or 2) DNA residues. The number of residues within each unit may be the same or variable from one unit to another. The oligonucleotide may have an odd or an even number of units. The oligonucleotide may start (i.e. at its 5′ end) with either a sugar-modified nucleotide-containing unit (e.g., a FANA-containing unit) or a DNA-containing unit. The oligonucleotide may terminate (i.e. at its 3′ end) with either a sugar-modified nucleotide-containing unit or a DNA-containing unit. The total number of units may be as few as 4 (i.e. at least 2 of each type).

In some embodiments, a FANA antisense oligonucleotide disclosed herein comprises alternating segments or units of arabinonucleotides and 2′-deoxynucleotides, wherein the segments or units each independently comprise at least one arabinonucleotide or 2′-deoxynucleotide, respectively. In some embodiments, the segments each independently comprise 1 to 2 arabinonucleotides or 2′-deoxynucleotides. In some embodiments, the segments each independently comprise 2 to 5 or 3 to 4 arabinonucleotides or 2′-deoxynucleotides. In some embodiments, a FANA antisense oligonucleotide disclosed herein comprises alternating segments or units of arabinonucleotides and 2′-deoxynucleotides, wherein the segments or units each comprise one arabinonucleotide or 2′-deoxynucleotide, respectively. In some embodiments, the segments each independently comprise about 3 arabinonucleotides or 2′-deoxynucleotides. In some embodiments, a FANA antisense oligonucleotide disclosed herein comprises alternating segments or units of arabinonucleotides and 2′-deoxynucleotides, wherein the segments or units each comprise one arabinonucleotide or 2′-deoxynucleotide, respectively. In some embodiments, a FANA antisense oligonucleotide disclosed herein comprises alternating segments or units of arabinonucleotides and 2′-deoxynucleotides, wherein said segments or units each comprise two arabinonucleotides or 2′-deoxynucleotides, respectively.

In some embodiments, a FANA antisense oligonucleotides disclosed herein has a structure selected from:

a) (Aₓ-D_{y})ₙ I

b) (D_{y}-Aₓ)ₙ II

c) (Aₓ-D_{y})ₘ-Aₓ-D_{y}-Aₓ III

d) (D_{y}-Aₓ)ₘ-D_{y}-Aₓ-D_{y} IV

wherein each of m, x and y are each independently an integer greater than or equal to 1, n is an integer greater than or equal to 2, A is a sugar-modified nucleotide and D is a 2′-deoxyribonucleotide.

For example, a FANA antisense oligonucleotide disclosed herein has structure I wherein x=1, y=1 and n=10, thereby having a structure:
A-D-A-D-A-D-A-D-A-D-A-D-A-D-A-D-A-D-A-D.

In another example, a FANA antisense oligonucleotide disclosed herein has structure II wherein x=1, y=1 and n=10, thereby having a structure:
D-A-D-A-D-A-D-A-D-A-D-A-D-A-D-A-D-A-D-A.

In another example, a FANA antisense oligonucleotide disclosed herein has structure III wherein x=1, y=1 and n=9, thereby having a structure:
A-D-A-D-A-D-A-D-A-D-A-D-A-D-A-D-A-D-A-D-A.

In another example, a FANA antisense oligonucleotide disclosed herein has structure IV wherein x=1, y=1 and n=9, thereby having a structure:
D-A-D-A-D-A-D-A-D-A-D-A-D-A-D-A-D-A-D-A-D.

In another example, a FANA antisense oligonucleotide disclosed herein has structure I wherein x=2, y=2 and n=5, thereby having a structure:
A-A-D-D-A-A-D-D-A-A-D-D-A-A-D-D-A-A-D-D.

In another example, a FANA antisense oligonucleotide disclosed herein has structure II wherein x=2, y=2 and n=5, thereby having a structure:
D-D-A-A-D-D-A-A-D-D-A-A-D-D-A-A-D-D-A-A.

In another example, a FANA antisense oligonucleotide disclosed herein has structure III wherein x=2, y=2 and m=4, thereby having a structure:
A-A-D-D-A-A-D-D-A-A-D-D-A-A-D-D-A-A-D-D-A-A.

In another example, a FANA antisense oligonucleotide disclosed herein has structure IV wherein x=2, y=2 and m=4, thereby having a structure:
D-D-A-A-D-D-A-A-D-D-A-A-D-D-A-A-D-D-A-A-D-D.

The antisense oligonucleotides of the disclosure include at least one FANA modified nucleotide. The antisense oligonucleotide sequence may include a modified sugar moiety for all or only a portion of the nucleotides in the sequence. In some embodiments, the antisense oligonucleotides may have all modified sugar moiety nucleotides in the sequence. In some embodiments, the antisense oligonucleotides may have 1, 2, 3, 4, 5, 6, 7, 8, 9, 10, 11, 12, 13, 14, 15, 16, 17, 18, 19, 20, or more than 20 unmodified nucleotides. In certain embodiments, the antisense oligonucleotide includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 FANA modified nucleotides. In certain other embodiments, the antisense oligonucleotide of the disclosure includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 FANA modified nucleotides.

In some embodiments, at least one unmodified nucleotide is located in the antisense oligonucleotide between strings of nucleotides which have modified sugar moieties. For example, an antisense oligonucleotide may have a string of at least 2, at least 3, at least 4, at least 5, at least 6, at least 7, at least 8, at least 9, at least 10 or more FANA modified nucleotides, followed by a string of at least 1, at least 2, at least 3, at least 4, at least 5, at least 6, at least 7, at least 8, at least 9, at least 10, or more unmodified nucleotides, followed by another string of at least 2, at least 3, at least 4, at least 5, at least 6, at least 7, at least 8, at least 9, at least 10 or more FANA modified nucleotides. In certain embodiments, when one or more unmodified nucleotides are flanked by FANA modified nucleotides, the unmodified nucleotide section may be referred to as a “nucleotide gap sequence.” The gap sequence may consist of 1, 2, 3, 4, 5, 6, 7, 8, 9, 10, 11, 12, 13, 14, 15, 16, 17, 18, 19, 20, or more than 20 unmodified nucleotides. In some embodiments, the antisense oligonucleotide may have a single gap sequence or may have more than one nucleotide gap sequence in the same molecule. The string of FANA modified nucleotides on each side of the unmodified nucleotide gap sequence can be of the same or of different lengths. For example, the antisense oligonucleotide may have 8 FANA modified nucleotides, followed by 7 unmodified nucleotides, followed by a second string of FANA modified nucleotides that is the same or different in number of FANA modified nucleotides as the first modified string. In certain embodiments the antisense oligonucleotide consists of FANA sugar modified nucleotides sequences flanking a gap sequence of unmodified nucleotides. For example, the antisense oligonucleotide comprises a FANA modified sequence between 1 and 10 nucleotides in length, then an unmodified nucleotide sequence between 1 and 10 nucleotides in length, followed by another FANA modified sequence between 1 and 10 nucleotides in length, with this pattern of modified and unmodified nucleotides optionally repeating.

Table 1 shows exemplary FANA nucleoside placement within 21-mer FANA antisense oligonucleotide. In some embodiments, FANA modified nucleotides are positioned according to any of Formulas 1-16.

TABLE 1

| 21 nucleotides | Formula |
|---|---|
| Formula 1 | XXXXXXXXXXXXXXXXXXXXX |
| Formula 2 | XXXXXXXXXXXXXXXXXXXXX |
| Formula 3 | XXXXXXXXXXXXXXXXXXXXX |
| Formula 4 | XXXXXXXXXXXXXXXXXXXXX |
| Formula 5 | XXXXXXXXXXXXXXXXXXXXX |
| Formula 6 | XXXXXXXXXXXXXXXXXXXXX |
| Formula 7 | XXXXXXXXXXXXXXXXXXXXX |
| Formula 8 | XXXXXXXXXXXXXXXXXXXXX |
| Formula 9 | XXXXXXXXXXXXXXXXXXXXX |
| Formula 10 | XXXXXXXXXXXXXXXXXXXXX |
| Formula 11 | XXXXXXXXXXXXXXXXXXXXX |
| Formula 12 | XXXXXXXXXXXXXXXXXXXXX |
| Formula 13 | XXXXXXXXXXXXXXXXXXXXX |
| Formula 14 | XXXXXXXXXXXXXXXXXXXXX |
| Formula 15 | XXXXXXXXXXXXXXXXXXXXX |
| Formula 16 | XXXXXXXXXXXXXXXXXXXXX |

The formulas shown in Table 1 can be applied to any sequence of the disclosure (e.g., the sequences set forth in SEQ ID NOs 533-567), wherein X represents an unmodified nucleotide and bolded and underlined nucleotides represent a FANA modified nucleotide.

In certain embodiments, the antisense oligonucleotides may be between 1 and 60 nucleotides long. Embodiments of this disclosure include antisense oligonucleotides having a length of 18-25 nucleotides (e.g., 18-mers, 19-mers, 20-mers, 21-mers, 22-mers, 23-mers, 24-mers, or 25-mers), or antisense oligonucleotides having a length of 26 or more nucleotides (e.g., 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides).

Antisense oligonucleotides as described herein include at least one segment of 18 or more contiguous nucleotides that are complementary to a fragment of equivalent size of the DNA of a target gene or the RNA transcript of the target gene.

In certain embodiments, the antisense oligonucleotides target sequences in the transcript of essential genes in specific weed species. Binding of the antisense oligonucleotide to mRNA is followed by RNase H cleavage of the mRNA transcript, resulting in reduced transcript abundance. Sufficient reduction in transcript abundance through this process results in loss of essential protein activity and ultimately plant death.

In certain embodiments, the target sequence may be specific to a single species. In certain embodiments, the targeted sequence may have activity across all species in a genus or family. Control of multiple species within a single application may be achieved by use of antisense oligonucleotides that match targeted transcripts across all species, or by combination of multiple antisense oligonucleotides targeting the transcript of one gene with sequence specificity for each respective species.

### Target Genes

Compositions and methods of the disclosure are useful for reducing the expression of a target gene in a plant cell. Compositions of the disclosure can include antisense oligonucleotides designed to target multiple genes, or multiple segments of one or more genes. The target can include multiple consecutive segments of a target gene, multiple non-consecutive segments of a target gene, multiple alleles of a target gene, or multiple target genes from one or more species.

Target genes can include essential genes, which are genes necessary for sustaining cellular life or to support reproduction of an organism. Embodiments of essential genes include genes involved in branched chain amino acid synthesis, photosynthesis, aromatic amino acid synthesis, lipid synthesis, sphingolipid synthesis, terpenoid synthesis, lysine synthesis, and tubulin synthesis.

In certain embodiments, the target gene is involved in branched chain amino acid synthesis. Gene targets for branched chain amino acid synthesis include, but are not limited to, the catalytic subunit of acetolactate of synthase (ALS) (also known as acetohydroxyacid synthease (AHAS)), the regulatory subunit of ALS, ketol-acid reductoisomerase (KARI), and dihydroxyacid dehydratase (DHAD).

In certain embodiments, the essential gene is involved in photosynthesis. Gene targets for photosynthesis include, but are not limited to, gamma subunit of ATP synthase (ATPC1), protoporphyrinogen oxidase, glutamine synthase (GS), phytoene desaturase (PDS), 4-hydroxyphenylpyruvate dioxygenase (HPPD), D1 protein of photosystem II (psbA), a ribulose-bisphosphate carboxylase (Rubisco). Inhibition of GS disrupts the carboxylase and oxygenase activity of Rubisco, a major sink for chemical energy (NADPH and ATP) produced in the light reactions of photosynthesis. The excess of electrons overwhelms the antioxidant system and is accepted by molecular oxygen leading to oxidative stress and rapid cell death. ATPC1 is the gamma subunit of ATP synthase (ATPase) and is important in regulating flow of protons through complex. The combination of inhibiting multiple targets generates more reactive oxygen species (ROS), oxidative stress, and cell death.

In certain embodiments, the target gene is involved in aromatic amino acid synthesis. Gene targets for aromatic amino acid synthesis include, but are not limited to, 3-deoxy-d-arabino-heptulosonate 7-phosphate synthase (DAHPS) and 5-enolpyruvylshikimate 3-phosphate synthase (EPSPS; also referred to as 3-phosphoshikimate 1-carboxyvinyltransferase). DAHPS is the committed enzymatic step for entry to the pathway and the regulatory control point. Inhibiting both DAHPS and EPSPS eliminates chorismite production, aromatic amino acids, and approximately 20% of total carbon flux in the plant. Plants typically have two to three genes for DAHPS and one to two genes for EPSPS.

In certain embodiments, the target gene is involved in lipid synthesis. Gene targets for lipid synthesis include, but are not limited to, acetyl-CoA carboxylase (ACCase) including both chloroplastic and cytoplasmic isoforms and both alpha and beta subunits in dicot species, fatty acid thioesterases (FATA or FATB), and 3-keto-acyl-CoA synthase, which is a very long chain fatty acid (VLCFA) elongase. Combinations of these targets are expected to greatly reduce plant synthesis of lipids for weed control.

In certain embodiments, the target gene is involved in sphingolipid synthesis. Gene targets for sphingolipid synthesis include, but are not limited to, ceramide synthases (e.g., LOH1 and LOH3). These are known targets of fungal mycotoxins (e.g., FB1, AAL) that cause programmed cell death in plants by inhibiting ceramide synthase that causes accumulation of sphingolipids (sphinganine, sphingoid bases). Accumulation of sphingolipids is highly toxic to plants. Double knockouts of LOH1/LOH3 result in programmed cell death, mimicking application of FB1 or AAL toxins.

In certain embodiments, the target gene is involved in terpenoid synthesis. Gene targets for terpenoid synthesis include, but are not limited to, mevalonate kinase. Mevalocidin is a fungal inhibitor of mevalonate kinase that causes lethality in both grass and dicot weeds.

In certain embodiments, the target gene is involved in lysine biosynthesis. Plants utilize the diaminopimelate (DAP) pathway, a branch of the aspartate-derived super-pathway, to synthesize L-lysine. L-aspartate semialdehyde (ASA) and pyruvate are converted to (4S)-4-hydroxy-2,3,4,5-tetrahydro-(2S)-dipicolinic acid (HTPA) in a condensation reaction catalyzed by dihydrodipicolinate synthase (DHDPS). Dihydrodipicolinate reductase (DHDPR) then catalyzes an NAD (P) H-dependent reduction of HTPA to produce 2,3,4,5-tetrahydrodipicolinate (THDP). THDP subsequently undergoes an amino-transfer reaction with L-glutamate, catalyzed by diaminopimelate aminotransferase (DAPAT), to yield LL-DAP. LL-DAP is converted to meso-DAP by diaminopimelate epimerase (DAPEpi) and lastly, meso-DAP is decarboxylated by diaminopimelate decarboxylase (DAPDC) to yield L-lysine, which imparts a negative feedback loop on DHDPS. Suitable gene targets include, but are not limited to, DHDPS and DHDPR, either alone or in combination, to eliminate lysine biosynthesis in target weeds.

In certain embodiments, the target gene is involved in tubulin synthesis. Gene targets for tubulin include, but are not limited to, alpha-tubulin, thereby inhibiting the assembly of microtubule chains. Alpha-tubulin is encoded by multiple genes in plants, usually four to six genes.

In certain embodiments, the target gene is involved in the C4 pathway of photosynthesis. Gene targets for the C4 pathway include, but are not limited to, phosphoenolpyruvate carboxylase (PEPC) and pyruvate orthophosphate dikinase (PPDK). These genes are essential for photosynthesis in C4 plants but are either absent or not essential in C3 plants, thereby enabling selective control of C4 weeds in C3 crops, such as controlling Palmer amaranth in soybean fields.

Examples of nucleotide and amino sequences of essential genes from the weed species Amaranthus palmeri (Ap), Lolium rigidum (Lr), Bassia scoparia (Bs), and Alopecurus myosuroides (Am) are summarized in Tables 2 and 3. Sequences of the genes from Arabidopsis thaliana (At), Zea mays (Zm), Glycine max (Gm), and Triticum aestivum (Ta) are also provided.

TABLE 2

| | | Coding Sequence SEQ ID NO: | | | | | | | |
|---|---|---|---|---|---|---|---|---|---|
| | Gene Name | At | Ap | Lr | Bs | Am | Zm | Gm | Ta |
| ALS branched | Acetolactate synthase, catalytic | 279 | 1 | 69 | 135 | 201, 202 | 353 | 415 | 477 |
| chain pathway | subunit (ALS) | | | | | | | | |
| | Acetolactate synthase, regulatory | 280 | 2 | 70 | 136 | 203 | 354 | 416 | 478 |
| | (small) subunit 1 | | | | | | | | |
| | Acetolactate synthase, regulatory | 281 | 3 | 71 | 137 | 204 | — | — | — |
| | (small) subunit 2 | | | | | | | | |
| | Ketol-acid reductoisomerase (KARI) | 282 | 4 | 72 | 138 | 205, 206 | 355 | 417 | 479 |
| | Dihydroxyacid dehydratase (DHAD) | 283 | 5 | 73 | 139 | 207 | 356 | 418 | 480 |
| Photosynthesis/ROS | CF1 ATP synthase (ATPC1) | 284 | 6 | 74 | 140 | 208, 209 | 357 | 419 | 481 |
| | Protoporphyrinogen oxidase 1 (PPO1, | 285 | 7 | 75 | 141 | 210, 211 | 358 | 420 | 482 |
| | PPX1) | | | | | | | | |
| | Protoporphyrinogen oxidase 2 (PPO2, | 286 | 8 | 76 | 142 | 212 | 359 | 421 | 483 |
| | PPX2) | | | | | | | | |
| | Glutamine synthetase 2 (GS2), | 287 | 9 | 77 | 143 | 213 | 360 | 422 | 484 |
| | chloroplastic | | | | | | | | |
| | Phytoene desaturase (PDS) | 288 | 10 | 78 | 144 | 214 | 361 | 423 | 485 |
| | 4-hydroxyphenylpyruvate | 289 | 11 | 79 | 145 | 215 | 362 | 424 | 486 |
| | dioxygenase (HPPD) | | | | | | | | |
| | Photosystem II Reaction Center | 290 | 12 | 80 | 146 | 216 | 363 | 425 | 487 |
| | Protein A, psbA | | | | | | | | |
| | Ribulose bisphosphate carboxylase | 291 | 13 | 81 | 147 | 217 | 364 | 426 | 488 |
| | large chain (Rubisco) | | | | | | | | |
| | CF1 ATP synthase (ATPC2) | 292 | 14 | — | 148 | | — | — | — |
| Aromatic | 5-enolpyruvylshikimate 3-phosphate | 293 | 15 | 82 | 149 | 218 | 365 | 427 | 489 |
| amino acid | synthase (EPSPS) | | | | | | | | |
| pathway | 3-deoxy-d-arabino-heptulosonate 7- | 294 | — | 83 | 150 | 219 | 366 | 428 | 490 |
| | phosphate synthase, DAHPS1 | | | | | | | | |
| | 3-deoxy-d-arabino-heptulosonate 7- | 295 | 16 | 84 | 151 | 220 | 367 | 429 | 491 |
| | phosphate synthase, DAHPS2 | | | | | | | | |
| | 3-deoxy-d-arabino-heptulosonate 7- | 296 | 17 | 85 | — | 221 | — | — | — |
| | phosphate synthase, DAHPS3 | | | | | | | | |
| Lipid synthesis | Acetyl-CoA Carboxylase (ACCase) | 297 | 18 | 86 | 152 | 222 | 368 | 430 | 492 |
| pathway | cytoplasmic | | | | | | | | |
| | Acetyl-CoA Carboxylase (ACCase) | 298 | 19 | 87 | — | 223 | — | 431 | — |
| | chloroplastic | | | | | | | | |
| | Acetyl-CoA Carboxylase (ACCase) | 299 | 20 | — | 153 | — | — | 432 | — |
| | carboxyltransferase alpha subunit | | | | | | | | |
| | Acetyl-CoA Carboxylase (ACCase) | 300 | 21 | — | — | — | — | 433 | — |
| | carboxyltransferase beta subunit | | | | | | | | |
| | Fatty acyl-ACP thioesterase A (FAT- | 301 | 22 | 88 | 154 | 224 | 369 | 434 | 493 |
| | A) | | | | | | | | |
| | Fatty acyl-ACP thioesterase B (FAT- | 302 | 23 | 89 | 155 | 225, | 370 | 435 | 494 |
| | B) | | | | | 226 | | | |
| | 3-ketoacyl-CoA synthase 1 | 303 | 24 | 90 | 156 | 227 | 371 | 436 | 495 |
| | (VLCFAE) | | | | | | | | |
| Sphingolipids | Ceramide synthase (LOH1) | 304 | 25 | 91 | 157 | 228 | 372 | 437 | 496 |
| | Ceramide synthase (LOH3) | 305 | — | 92 | 158 | 229 | 373 | — | 497 |
| Terpenoid | Mevalonate kinase | 306 | 26 | 93 | 159 | 230 | 374 | 438 | 498 |
| synthesis | | | | | | | | | |
| Lysine | Dihydrodipicolinate synthase | 307 | 27 | 94 | 160 | 231 | 375 | 439 | 499 |
| synthesis | (DHDPS1) | | | | | | | | |
| | Dihydrodipicolinate synthase | 308 | 28 | 95 | 161 | — | — | — | — |
| | (DHDPS2) | | | | | | | | |
| | Dihydrodipicolinate reductase | 309 | 29 | 96 | 162 | 232 | 376 | 440 | 500 |
| | (DHDPR1) | | | | | | | | |
| | Dihydrodipicolinate reductase | 310 | 30 | 97 | — | 233 | — | 441 | 501 |
| | (DHDPR2) | | | | | | | | |
| Tubulin | Tubulin alpha-1 | 311 | 31 | 98 | 163 | 234 | 377 | 442 | 502 |
| synthesis | Tubulin alpha-2 | 312 | 32 | 99 | 164 | 235 | 378 | 443 | 503 |
| | Tubulin alpha-3 | 313 | — | 100 | 165 | 236 | 379 | 444 | 504 |
| | Tubulin alpha-6 | 314 | — | 101 | — | 237 | 380 | 445 | — |
| C4 specific | PEP carboxylase | — | 33 | — | 166 | 238 | 381 | — | — |
| | Pyruvate orthophosphate dikinase | 315 | 34 | — | 167 | 239 | 382, 383 | — | — |
| | (PPDK) | | | | | | | | |

TABLE 3

| | | Protein Sequence SEQ ID NO: | | | | | | | |
|---|---|---|---|---|---|---|---|---|---|
| | Gene Name | At | Ap | Lr | Br | Am | Zm | Gm | Ta |
| ALS branched | Acetolactate synthase, catalytic | 316 | 35 | 102 | 168 | 240, 241 | 384 | 446 | 505 |
| chain pathway | subunit (ALS) | | | | | | | | |
| | Acetolactate synthase, regulatory | 317 | 36 | 103 | 169 | 242 | 385 | 447 | 506 |
| | (small) subunit 1 | | | | | | | | |
| | Acetolactate synthase, regulatory | 318 | 37 | 104 | 170 | 243 | — | — | — |
| | (small) subunit 2 | | | | | | | | |
| | Ketol-acid reductoisomerase | 319 | 38 | 105 | 171 | 244 | 386 | 448 | 507 |
| | (KARI) | | | | | 245 | | | |
| | Dihydroxyacid dehydratase | 320 | 39 | 106 | 172 | 246 | 387 | 449 | 508 |
| | (DHAD) | | | | | | | | |
| Photosynthesis/ | CF1 ATP synthase (ATPC1) | 321 | 40 | 107 | 173 | 247, 248 | 388 | 450 | 509 |
| ROS | Protoporphyrinogen oxidase 1 | 322 | 41 | 108 | 174 | 249, 250 | 389 | 451 | 510 |
| | (PPO1, PPX1) | | | | | | | | |
| | Protoporphyrinogen oxidase 2 | 323 | 42 | 109 | 175 | 251 | 390 | 452 | 511 |
| | (PPO2, PPX2) | | | | | | | | |
| | Glutamine synthetase 2 (GS2), | 324 | 43 | 110 | 176 | 252 | 391 | 453 | 512 |
| | chloroplastic | | | | | | | | |
| | Phytoene desaturase (PDS) | 325 | 44 | 111 | 177 | 253 | 392 | 454 | 513 |
| | 4-hydroxyphenylpyruvate | 326 | 45 | 112 | 178 | 254 | 393 | 455 | 514 |
| | dioxygenase (HPPD) | | | | | | | | |
| | Photosystem II Reaction Center | 327 | 46 | 113 | 179 | 255 | 394 | 456 | 515 |
| | Protein A, psbA | | | | | | | | |
| | Ribulose bisphosphate carboxylase | 328 | 47 | 114 | 180 | 256 | 395 | 457 | 516 |
| | large chain (Rubisco) | | | | | | | | |
| | CF1 ATP synthase (ATPC2) | 329 | 48 | — | 181 | 39 | — | — | — |
| Aromatic | 5-enolpyruvylshikimate 3-phosphate | 330 | 49 | 115 | 182 | 257 | 396 | 458 | 517 |
| amino acid | synthase (EPSPS) | | | | | | | | |
| pathway | 3-deoxy-d-arabino-heptulosonate 7- | 331 | — | 116 | 183 | 258 | 397 | 459 | 518 |
| | phosphate synthase, DAHPS1 | | | | | | | | |
| | 3-deoxy-d-arabino-heptulosonate 7- | 332 | 50 | 117 | 184 | 259 | 398 | 460 | 519 |
| | phosphate synthase, DAHPS2 | | | | | | | | |
| | 3-deoxy-d-arabino-heptulosonate 7- | 333 | 51 | 118 | — | 260 | — | — | — |
| | phosphate synthase, DAHPS3 | | | | | | | | |
| Lipid synthesis | Acetyl-CoA Carboxylase (ACCase) | 334 | 52 | 119 | 185 | 261 | 399 | 461 | 520 |
| pathway | cytoplasmic | | | | | | | | |
| | Acetyl-CoA Carboxylase (ACCase) | 335 | 53 | 120 | — | 262 | — | 462 | — |
| | chloroplastic | | | | | | | | |
| | Acetyl-CoA Carboxylase (ACCase) | 336 | 54 | — | 186 | — | — | 463 | — |
| | carboxyltransferase alpha subunit | | | | | | | | |
| | Acetyl-CoA Carboxylase (ACCase) | 337 | 55 | — | — | — | — | 464 | — |
| | carboxyltransferase beta subunit | | | | | | | | |
| | Fatty acyl-ACP thioesterase A | 338 | 56 | 121 | 187 | 263 | 400 | 465 | 521 |
| | (FAT-A) | | | | | | | | |
| | Fatty acyl-ACP thioesterase B | 339 | 57 | 122 | 188 | 264, 265 | 401 | 466 | 522 |
| | (FAT-B) | | | | | | | | |
| | 3-ketoacyl-CoA synthase 1 | 340 | 58 | 123 | 189 | 266 | 402 | 467 | 523 |
| | (VLCFAE) | | | | | | | | |
| Sphingolipids | Ceramide synthase (LOH1) | 341 | 59 | 124 | 190 | 267 | 403 | 468 | 524 |
| | Ceramide synthase (LOH3) | 342 | — | 125 | 191 | 268 | 404 | — | 525 |
| Terpenoid | Mevalonate kinase | 343 | 60 | 126 | 192 | 269 | 405 | 469 | 526 |
| synthesis | | | | | | | | | |
| Lysine | Dihydrodipicolinate synthase | 344 | 61 | 127 | 193 | 270 | 406 | 470 | 527 |
| synthesis | (DHDPS1) | | | | | | | | |
| | Dihydrodipicolinate synthase | 345 | 62 | 128 | 194 | — | — | — | — |
| | (DHDPS2) | | | | | | | | |
| | Dihydrodipicolinate reductase | 346 | 63 | 129 | 195 | 271 | 407 | 471 | 528 |
| | (DHDPR1) | | | | | | | | |
| | Dihydrodipicolinate reductase | 347 | 64 | 130 | — | 272 | — | 472 | 529 |
| | (DHDPR2) | | | | | | | | |
| Tubulin | Tubulin alpha-1 | 348 | 65 | 131 | 196 | 273 | 408 | 473 | 530 |
| synthesis | Tubulin alpha-2 | 349 | 66 | 132 | 197 | 274 | 409 | 474 | 531 |
| | Tubulin alpha-3 | 350 | — | 133 | 198 | 275 | 410 | 475 | 532 |
| | Tubulin alpha-6 | 351 | — | 134 | — | 276 | 411 | 476 | — |
| C4 specific | PEP carboxylase | — | 67 | — | 199 | 277 | 412 | — | — |
| | Pyruvate orthophosphate dikinase | 352 | 68 | — | 200 | 278 | 413, 414 | — | — |
| | (PPDK) | | | | | | | | |

Those skilled in the art may also find target genes in additional plant species based on genome synteny and sequence similarity. In one embodiment, additional target genes can be obtained by hybridization or PCR using sequences based on the nucleotide sequences noted above.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as 32P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

In certain embodiments, the target gene comprises a sequence having at least 80%, at least 90%, at least 95%, at least 96%, at least 97% at least 98%, at least 99%, at least 99.5%, or 100% sequence identity to at least one of SEQ ID NOs: 1-34, 69-101, 135-167, 201-239, 279-315, 353-383, 415-445, 477-504.

In certain embodiments, the target gene encodes a polypeptide having at least 80%, at least 90%, at least 95%, at least 96%, at least 97% at least 98%, at least 99%, at least 99.5%, or 100% sequence identity to at least one of SEQ ID NOs: 35-68, 102-134, 168-200, 240-278, 316-352, 384-414, 446-476, 505-532.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. In another embodiment, the percent identity is calculated across the entirety of the reference sequence. The percent identity between two sequences can be determined using techniques similar to those described below, with or without allowing gaps. In calculating percent identity, typically exact matches are counted. A gap, i.e. a position in an alignment where a residue is present in one sequence but not in the other, is regarded as a position with non-identical residues.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to target genes of the disclosure. BLAST protein searches can be performed with the BLASTX program, score=50, wordlength=3, to obtain amino acid sequences homologous to proteins encoded by the target genes of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). When utilizing the ALIGN program for comparing amino acid sequences, a PAM120 weight residue table, a gap length penalty of 12, and a gap penalty of 4 can be used.

### Antisense Oligonucleotide Compositions

The antisense oligonucleotide compositions used in the embodiments disclosed herein comprise at least one FANA antisense oligonucleotide (e.g., two or more, such as two, three, four, five, six, seven, eight, nine, ten, etc.). For their use in practice, the antisense oligonucleotides of the disclosure can be used alone and can also advantageously be used in formulations in combination or association with one or more other compatible components, which are suitable for the desired use and which are acceptable for use in agriculture. The formulations can be of any type known in the art which are suitable for application onto all types of crops. The compositions disclosed herein may be formulated for various agricultural applications (e.g., seed coating formulations, foliar applications, in-furrow applications, drench applications, etc.). The compositions described herein may be formulated with one or more suitable auxiliaries to achieve a particular purpose (e.g., to coat seeds, for foliar applications, for dilution, etc.). These formulations, which can be prepared in any manner known in the art, also form part of the disclosure.

In certain embodiments, the formulation comprises at least one antisense oligonucleotide of the disclosure and at least one agriculturally suitable auxiliary, e.g., a carrier or a surfactant.

A carrier is a solid or liquid, natural or synthetic, organic or inorganic substance that is generally inert. The carrier generally improves the application of the antisense oligonucleotide, for instance, to plants, plants parts or seeds. Examples of suitable solid carriers include, but are not limited to, ammonium salts, in particular ammonium sulfates, ammonium phosphates and ammonium nitrates, natural rock flours, such as kaolins, clays, talc, chalk, quartz, attapulgite, montmorillonite and diatomaceous earth, silica gel and synthetic rock flours, such as finely divided silica, alumina and silicates. Examples of typically useful solid carriers for preparing granules include, but are not limited to crushed and fractionated natural rocks such as calcite, marble, pumice, sepiolite and dolomite, synthetic granules of inorganic and organic flours and granules of organic material such as paper, sawdust, coconut shells, maize cobs and tobacco stalks. Examples of suitable liquid carriers include, but are not limited to, water, organic solvents and combinations thereof. Examples of suitable solvents include polar and nonpolar organic chemical liquids, for example from the classes of aromatic and nonaromatic hydrocarbons (such as cyclohexane, paraffins, alkylbenzenes, xylene, toluene, tetrahydronaphthalene, alkylnaphthalenes, chlorinated aromatics or chlorinated aliphatic hydrocarbons such as chlorobenzenes, chloroethylenes or methylene chloride), alcohols and polyols (which may optionally also be substituted, etherified and/or esterified, such as ethanol, propanol, butanol, benzylalcohol, cyclohexanol or glycol), ketones (such as acetone, methyl ethyl ketone, methyl isobutyl ketone, acetophenone, or cyclohexanone), esters (including fats and oils) and (poly) ethers, unsubstituted and substituted amines, amides (such as dimethylformamide or fatty acid amides) and esters thereof, lactams (such as N-alkylpyrrolidones, in particular N-methylpyrrolidone) and lactones, sulfones and sulfoxides (such as dimethyl sulfoxide), oils of vegetable or animal origin, nitriles (alkyl nitriles such as acetonitrile, propionotrilie, butyronitrile, or aromatic nitriles, such as benzonitrile), carbonic acid esters (cyclic carbonic acid esters, such as ethylene carbonate, propylene carbonate, butylene carbonate, or dialkyl carbonic acid esters, such as dimethyl carbonate, diethyl carbonate, dipropyl carbonate, dibutyl carbonate, dioctyl carbonate). The carrier may also be a liquefied gaseous extender, i.e. liquid which is gaseous at standard temperature and under standard pressure, for example aerosol propellants such as halohydrocarbons, butane, propane, nitrogen and carbon dioxide.

In certain embodiments, the amount of carrier ranges from 1% to 99.9%, from 5% to 99%, from 10% to 95%, or from 20% to 90% by weight of the composition.

Surfactants are well known in the art and any combination of suitable surfactants or surfactant systems may be used with the antisense oligonucleotide compositions described herein. The following includes non-limiting examples of surfactants which may be suitable for use with the antisense oligonucleotide compositions described herein.

The antisense oligonucleotide compositions described herein may comprise one or more anionic surfactants. The anionic surfactant(s) may be either water soluble anionic surfactants, water insoluble anionic surfactants, or a combination of water soluble anionic surfactants and water insoluble anionic surfactants. Mixtures of anionic and nonionic surfactants may also be used in the compositions.

Anionic surfactants are surfactants having a hydrophilic moiety in an anionic or negatively charged state in aqueous solution. Non-limiting examples of anionic surfactants include sulfonic acids, sulfuric acid esters, carboxylic acids, and salts thereof. Non-limiting examples of water soluble anionic surfactants include alkyl sulfates, alkyl ether sulfates, alkyl amido ether sulfates, alkyl aryl polyether sulfates, alkyl aryl sulfates, alkyl aryl sulfonates, monoglyceride sulfates, alkyl sulfonates, alkyl amide sulfonates, alkyl aryl sulfonates, benzene sulfonates, toluene sulfonates, xylene sulfonates, cumene sulfonates, alkyl benzene sulfonates, alkyl diphenyloxide sulfonate, alpha-olefin sulfonates, alkyl naphthalene sulfonates, paraffin sulfonates, lignin sulfonates, alkyl sulfosuccinates, ethoxylated sulfosuccinates, alkyl ether sulfosuccinates, alkylamide sulfosuccinates, alkyl sulfosuccinamate, alkyl sulfoacetates, alkyl phosphates, phosphate ester, alkyl ether phosphates, acyl sarconsinates, acyl isethionates, N-acyl taurates, N-acyl-N-alkyltaurates, alkyl carboxylates, or a combination thereof.

The antisense oligonucleotide compositions described herein may comprise one or more nonionic surfactants. The nonionic surfactant may be either water soluble nonionic surfactants, water insoluble nonionic surfactants, or a combination of water soluble nonionic surfactants and water insoluble nonionic surfactants.

Non-limiting examples of water insoluble nonionic surfactants include alkyl and aryl: glycerol ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, polyoxyethylenated polyoxyproylene glycols, sorbitan fatty esters, or combinations thereof. Also included are EO/PO block copolymers (EO is ethylene oxide, PO is propylene oxide), EO polymers and copolymers, polyamines, and polyvinylpynolidones. Commercially available water insoluble nonionic surfactants that may be suitable for the antisense oligonucleotide compositions described herein include Tomadol® 91-2.5, Tomadol® 23-1, Tomadol® 23-3, Span™ 20, Span™ 40, Span™ 60, Span™ 65, Span™ 80, Span™ 85, Arlatone® TV, Atlas® G-1086, Atlas® G-1096, Atlox® 1045A, Cirrasol® G-1086, Cirrasol® G-1096, and combinations thereof.

Non-limiting examples of water soluble nonionic surfactants include sorbitan fatty acid alcohol ethoxylates and sorbitan fatty acid ester ethoxylates. Commercially available water soluble nonionic surfactants that may be suitable for the antisense oligonucleotide compositions described herein include Tomadol® 9-11, Tomadol® 23-7, Tomadol® 91-6, Tween® 20, Tween® 21, Tween® 40, Tween® 60, Tween® 80, Surfonic L24-4, and combinations thereof.

In certain embodiments, the amount of surfactant ranges from 0.01% to 10%, from 0.05% to 5%, or from 0.1 to 1% by weight of the composition.

Antisense oligonucleotide compositions described herein may also comprise organosilicone surfactants, silicone-based antifoams used as surfactants in silicone-based and mineral-oil based antifoams. In another embodiment, the antisense oligonucleotide compositions described herein may also comprise alkali metal salts of fatty acids (e.g., water soluble alkali metal salts of fatty acids and/or water insoluble alkali metal salts of fatty acids).

Further examples of suitable auxiliaries include water repellents, drying agents, binders (adhesive, tackifier, fixing agent, such as carboxymethylcellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone and tylose), thickeners and secondary thickeners (such as cellulose ethers, acrylic acid derivatives, xanthan gum, modified clays, e.g., the products available under the name Bentone, and finely divided silica), stabilizers (e.g., cold stabilizers, preservatives (e.g., dichlorophene, benzyl alcohol hemiformal, 1,2-Benzisothiazolin-3-on, 2-methyl-4-isothiazolin-3-one), antioxidants, light stabilizers, in particular UV stabilizers, or other agents which improve chemical and/or physical stability), dyes or pigments (such as inorganic pigments, e.g., iron oxide, titanium oxide and Prussian Blue; organic dyes, e.g., alizarin, azo and metal phthalocyanine dyes), antifoams (e.g., silicone antifoams and magnesium stearate), antifreezes, stickers, gibberellins and processing auxiliaries, mineral and vegetable oils, perfumes, waxes, nutrients (including trace nutrients, such as salts of iron, manganese, boron, copper, cobalt, molybdenum and zinc), protective colloids, thixotropic substances, penetrants, sequestering agents and complex formers.

The antisense oligonucleotide compositions may be in any customary formulation type, such as solutions (e.g., aqueous solutions), emulsions, water- and oil-based suspensions, powders (e.g., wettable powders, soluble powders), dusts, pastes, granules (e.g., soluble granules, granules for broadcasting), suspoemulsion concentrates, natural or synthetic products impregnated with the antisense oligonucleotides, fertilizers and also microencapsulations in polymeric substances. The antisense oligonucleotides may be present in a suspended, emulsified or dissolved form. Examples of particular suitable formulation types are solutions, water-soluble concentrates, dispersible concentrates, suspensions and suspension concentrates, emulsifiable concentrates, emulsions, capsules, pastes, wettable powders or dusts, pressings, granules, as well as gel formulations for the treatment of plant propagation materials such as seeds.

### Nanoparticles

The antisense oligonucleotide compositions of the disclosure may include a nanoparticle to improve delivery and uptake into the plant.

As used herein, the term “nanoparticle” refers to particles having at least one cross-sectional dimension of less than about 1 micron. A nanoparticle can also be referred to as a “nanostructure.” A nanoparticle can have at least one cross-sectional dimension of less than about 500 nm, less than about 250 nm, less than about 100 nm, less than about 75 nm, less than about 50 nm, less than about 25 nm, less than about 10 nm, or, in some cases, less than about 1 nm. Examples of nanoparticles include nanotubes (e.g., carbon nanotubes), nanowires (e.g., carbon nanowires), graphene, star polycations, and quantum dots (e.g., carbon dots), among others.

A variety of nanoparticles can be used. Sometimes a nanoparticle can be a carbon-based nanoparticle. As used herein, a “carbon-based nanoparticle” can include a fused network of aromatic rings wherein the nanoparticle includes primarily carbon atoms. In some instances, a nanoparticle can have a cylindrical, pseudo-cylindrical, or horn shape. A carbon-based nanoparticle can include a fused network of at least about 10, at least about 50, at least about 100, at least about 1000, at least about 10,000, or, in some cases, at least about 100,000 aromatic rings. A carbon-based nanoparticle may be substantially planar or substantially non-planar, or may include a planar or non-planar portion. A carbon-based nanoparticle may optionally include a border at which the fused network terminates. For example, a sheet of graphene includes a planar carbon-containing molecule including a border at which the fused network terminates, while a carbon nanotube includes a non-planar carbon-based nanoparticle with borders at either end. In some cases, the border may be substituted with hydrogen atoms. In some cases, the border may be substituted with groups comprising oxygen atoms (e.g., hydroxyl).

In some embodiments, a nanoparticle can include or be a nanotube. The term “nanotube” is given its ordinary meaning in the art and can refer to a substantially cylindrical molecule or nanoparticle including a fused network of primarily six-membered rings (e.g., six-membered aromatic rings). In some cases, a nanotube can resemble a sheet of graphite formed into a seamless cylindrical structure. It should be understood that a nanotube may also include rings or lattice structures other than six-membered rings. Typically, at least one end of the nanotube may be capped, i.e., with a curved or non-planar aromatic group. A nanotube may have a diameter of the order of nanometers and a length on the order of microns, tens of microns, hundreds of microns, or millimeters, resulting in an aspect ratio greater than about 100, about 1000, about 10,000, or greater. In some embodiments, a nanotube can have a diameter of less than about 1 micron, less than about 500 nm, less than about 250 nm, less than about 100 nm, less than about 75 nm, less than about 50 nm, less than about 25 nm, less than about 10 nm, or, in some cases, less than about 1 nm.

In some embodiments, a nanotube may include a carbon nanotube. The term “carbon nanotube” can refer to a nanotube including primarily carbon atoms. Examples of carbon nanotubes can include single-walled carbon nanotubes (SWNTs), double-walled carbon nanotubes (DWNTs), multi-walled carbon nanotubes (MWNTs) (e.g., concentric carbon nanotubes), inorganic derivatives thereof, and the like. In some embodiments, a carbon nanotube can be a single-walled carbon nanotube. In some cases, a carbon nanotube can be a multi-walled carbon nanotube (e.g., a double-walled carbon nanotube).

The term “quantum dot” is given its normal meaning in the art. Examples of materials from which quantum dots can be made include PbS, PbSe, CdS, CdSe, ZnS, and ZnSe, among others. Carbon dots are quantum sized carbon nanoparticles which have emerged as being nanoparticles with potential to replace heavy metal containing toxic quantum dots. In certain embodiments, the nanoparticle comprises or is a carbon dot.

### Herbicides

In certain embodiments, the antisense oligonucleotide compositions described herein may optionally comprise one or more herbicides. Alternatively, the one or more herbicides may be applied either simultaneously or applied sequentially, with the antisense oligonucleotide compositions disclosed herein. In certain embodiments, the herbicide may be a pre-emergent herbicide, a post-emergent herbicide, or a combination thereof.

Non-limiting examples of suitable herbicides include bentazon, acifluorfen, chlorimuron, lactofen, clomazone, fluazifop, glufosinate, glyphosate, sethoxydim, imazethapyr, imazamox, fomesafe, flumiclorac, imazaquin, and clethodim. Commercial products containing each of these compounds are readily available. Herbicide concentration in the composition will generally correspond to the labeled use rate for a particular herbicide.

The antisense oligonucleotide compositions of the disclosure may be used to reduce the expression of herbicide resistance genes in weeds and restore utility of herbicides at originally labeled use rates on herbicide resistant weeds (e.g., genes for herbicide metabolism such as cytochrome P450s, glutathione-S-transferases, and aldo-keto reductase; target-site genes that are over-expressed such as EPSPS; and genes for altered herbicide movement such as ABC transporters).

### Application to Plants

All plants and plant parts can be treated in accordance with the disclosure. Here, plants are to be understood to mean all plants and plant parts such as wanted and unwanted wild plants or crop plants (including naturally occurring crop plants), for example cereals (wheat, rice, triticale, barley, rye, oats), maize, soybean, potato, sugar beet, sugar cane, tomatoes, pepper, cucumber, melon, carrot, watermelon, onion, lettuce, spinach, leek, beans, Brassica oleracea (e.g., cabbage) and other vegetable species, cotton, tobacco, oilseed rape, and also fruit plants (with the fruits apples, pears, citrus fruits and grapevines). Crop plants can be plants which can be obtained by conventional breeding and optimization methods or by biotechnological and genetic engineering methods or combinations of these methods. Plants should be understood to mean all developmental stages, such as seeds, seedlings, young (immature) plants up to mature plants. Plant parts should be understood to mean all parts and organs of the plants above and below ground, such as shoot, leaf, flower and root, examples given being leaves, needles, stalks, stems, flowers, fruit bodies, fruits and seeds, and also tubers, roots and rhizomes. Parts of plants also include harvested plants or harvested plant parts and vegetative and generative propagation material, for example seedlings, tubers, rhizomes, cuttings and seeds.

Methods of using the antisense oligonucleotide compositions of the disclosure to control one or more weed species are provided. Examples of weed species controlled using the methods described herein include, but are not limited to, Italian ryegrass (Lolium multiflorum), kochia (Kochia scoparia), redroot pigweed (Amaranthus retroflexus), palmer amaranth (Amaranthus palmeri), barnyardgrass (Echinochloa crus-galli), Common lambsquarters (Chenopodium album), Russian thistle (Salsola iberica), Common waterhemp (Amaranthus tuberculatus, syn. Amaranthus rudis), smooth pigweed (Amaranthus hybridus), powell amaranth (Amaranthus powellii), spiny amaranth (Amaranthus spinosus), black-grass (Alopecurus myosuroides), annual ryegrass (Lolium rigidum), perennial ryegrass (Lolium perenne), sourgrass (Digitaria insularis), junglerice (Echinochloa colona), Echinochloa phyllopogon, wild oats (Avena fatua), feral rye (Secale cereale), downy brome (Bromus tectorum), ripgut brome (Bromus diandrus), jointed goatgrass (Aegilops cylindrica), silky bentgrass (Apera spica-venti), green foxtail (Setaria viridis), yellow foxtail (Setaria pumila), eastern black nightshade (Solanum ptychanthum), horseweed (Conyza canadensis), hairy fleabane (Conyza bonariensis), tall fleabane (Conyza sumatrensis), broadleaf signalgrass (Urochloa platyphylla), giant foxtail (Setaria faberi), dandelion (Taraxacum officinale), blue mustard (Chorispora tenella), flixweed (Descurainia sophia), shepherd's-purse (Capsella bursa-pastoris), Sicklepod (Senna obtusifolia), hemp sesbania (Sesbania herbacea), velvetleaf (Abutilon theophrasti), johnsongrass (Sorghum halepense), giant ragweed (Ambrosia trifida), common ragweed (Ambrosia artemisiifolia), prickly sida (Sida spinosa), pitted morningglory, (Ipomoea lacunose), and puncturevine (Tribulus terrestris).

The treatment of the plants and plant parts with the antisense oligonucleotides of the disclosure is carried out directly or by action on their surroundings or habitat using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc.

A direct treatment of the plants is foliar application, i.e. the antisense oligonucleotides of the disclosure are applied to the foliage. The antisense oligonucleotides of the disclosure also access the plants via the root system. The plants are then treated by the action of the antisense oligonucleotides of the disclosure on the habitat of the plant. This may be done, for example, by drenching, or by mixing into the soil or the nutrient solution, i.e. the locus of the plant (e.g., soil) is impregnated with a liquid form of the antisense oligonucleotides of the disclosure, or by soil application, i.e. the antisense oligonucleotides of the disclosure are introduced in solid form (e.g., in the form of granules) into the locus of the plants, or by drip application (often also referred to as “chemigation”), i.e. the liquid application of the antisense oligonucleotides of the disclosure from surface or sub-surface driplines over a certain period of time together with varying amounts of water at defined locations in the vicinity of the plants.

Compositions of the disclosure can be applied to weeds, crops, soil, and any other desired target using any delivery methodology known to those of skill in the art. For example, antisense oligonucleotide compositions can be applied to the desired locale via methods and forms including, but not limited to, shank injection, sprays, granules, flood/furrow methods, sprinklers, fumigation, root soaking and drip irrigation. In embodiments of the disclosure where the compositions are sprayed onto a desired locale, the compositions can be delivered as a liquid suspension, emulsion, microemulsion or powder. In other embodiments, granules or microcapsules can be used to deliver the compositions of the disclosure.

The antisense oligonucleotide compositions of the disclosure can be applied to plants or crops by any convenient method, for example, by using a fixed application system such as a center pivot irrigation system. In certain embodiments, application to fields of plants or crops is made by air spraying, i.e., from an airplane or helicopter, or by land spraying. For example, land spraying may be carried out by using a high flotation applicator equipped with a boom, by a back-pack sprayer or by nurse trucks or tanks. One of skill in the art will recognize that these application methodologies are provided by way of example and that any applicable methods known in the art or developed in the future can be utilized.

In certain embodiments, a method for controlling one or more weeds with an antisense oligonucleotide comprises directly contacting a weed with one or more of the antisense oligonucleotides described herein. Non-limiting examples of contacting the weed include spraying the weed, drenching the weed, dripping onto the weed, or dusting the weed with one or more of the antisense oligonucleotides described herein. In one embodiment, the contacting step is repeated (e.g., more than once, as in the treating step is repeated twice, three times, four times, five times, six times, seven times, eight times, nine times, ten times, etc.). The contacting step can occur at any time during the growth of the weed. In one embodiment, contacting a weed with one or more of the antisense oligonucleotides described herein occurs before the weed begins to grow. In another embodiment, contacting a weed with one or more of the antisense oligonucleotides described herein occurs after the weed has started to grow.

In certain embodiments, a method for controlling one or more weeds with an antisense oligonucleotide comprises contacting a crop plant or plant part with one or more of the antisense oligonucleotides described herein. Non-limiting examples of contacting the crop plant or plant part include spraying the crop plant or plant part, drenching the crop plant or plant part, dripping onto the crop plant or plant part, or dusting the crop plant or plant part with one or more of the antisense oligonucleotides described herein. In one embodiment, the contacting step is repeated (e.g., more than once, as in the treating step is repeated twice, three times, four times, five times, six times, seven times, eight times, nine times, ten times, etc.). The contacting step can occur at any time during the growth of the crop plant or plant part. In one embodiment, contacting the crop plant or plant part with one or more of the antisense oligonucleotides described herein occurs before the crop plant begins to grow. In another embodiment, contacting the crop plant or plant part with one or more of the antisense oligonucleotides described herein occurs after the crop plant has started to grow.

In certain embodiments, a method for controlling one or more weeds with an antisense oligonucleotide comprises treating a soil with one or more of the antisense oligonucleotides described herein. Without being bound by theory, it is believed the one or more weeds will come into contact with the antisense oligonucleotides when in contact with a treated soil. Non-limiting examples of treating the soil include spraying the soil, drenching the soil, dripping onto the soil, and/or dusting the soil with one or more of the antisense oligonucleotide compositions described herein.

In another embodiment, the method further comprises the step of planting a plant or plant part in the soil. The planting step can occur before, after, or during the treatment of the soil with one or more of the antisense oligonucleotides described herein. In one embodiment, the planting step occurs before the soil is treated with one or more of the antisense oligonucleotide compositions described herein. In another embodiment, the planting step occurs during the treatment of the soil with one or more of the antisense oligonucleotide compositions described herein (e.g., the planting step occurs substantially simultaneously with the treating step, etc.). In still another embodiment, the planting step occurs after the soil is treated with one or more of the antisense oligonucleotide compositions described herein.

In certain embodiments, seeds are coated with one or more of the antisense oligonucleotide compositions described herein. Seeds may be treated with the compositions described herein in several ways including via spraying or dripping. Spray and drip treatment may be conducted by formulating antisense oligonucleotide compositions described herein and spraying or dripping the antisense oligonucleotide compositions onto seed via a continuous treating system (which is calibrated to apply treatment at a predefined rate in proportion to the continuous flow of seed), such as a drum-type of treater. Batch systems, in which a predetermined batch size of seed and antisense oligonucleotide compositions as described herein are delivered into a mixer, may also be employed. Systems and apparatuses for performing these processes are commercially available from numerous suppliers. In another embodiment, the treatment entails coating seeds. One such process involves coating the inside wall of a round container with the antisense oligonucleotide compositions described herein, adding seeds, then rotating the container to cause the seeds to contact the wall and the antisense oligonucleotide compositions, a process known in the art as container coating. Seeds can be coated by combinations of coating methods. Soaking typically entails using liquid forms of the antisense oligonucleotide compositions described. For example, seeds can be soaked for about 1 minute to about 24 hours (e.g., for at least 1 min, 5 min, 10 min, 20 min, 40 min, 80 min, 3 hours, 6 hours, 12 hours, 24 hours).

### Embodiments

The following numbered embodiments also form part of the present disclosure:

1. A method of controlling weeds, the method comprising: providing to the weed a composition comprising an antisense oligonucleotide comprising at least one FANA-modified nucleotide, wherein the antisense oligonucleotide targets a transcript of an essential gene of the weed.

2. The method of embodiment 1, wherein the antisense oligonucleotide comprises a sequence complementary to at least 18, at least 19, at least 20, at least 21, at least 22, at least 23, at least 24, or at least 25 contiguous nucleotides of the transcript.

3. The method of embodiment 1 or embodiment 2, wherein the antisense oligonucleotide comprises or consists of a sequence complementary to at least 18, at least 19, at least 20, at least 21, at least 22, at least 23, at least 24, or at least 25 contiguous nucleotides of at least one of SEQ ID NOs: 1-34, 69-101, 135-167, 201-239.

4. The method of any one of embodiments 1-3, wherein the antisense oligonucleotide comprises or consists of a sequence complementary to 21 contiguous nucleotides of at least one of SEQ ID NOs: 1-34, 69-101, 135-167, 201-239.

5. The method of any one of embodiments 1-4, wherein the antisense oligonucleotide comprises or consists of the sequence of at least one of SEQ ID NOs: 533-567.

6. The method of any one of embodiments 1-5, wherein the essential gene encodes a catalytic subunit of an acetolactate synthase, a regulatory subunit of an acetolactate synthase, a ketol-acid reductoisomerase, a dihydroxy-acid dehydratase, a gamma subunit of an ATP synthase, a protoporphyrinogen oxidase, a glutamine synthase, a phytoene desaturase, a 4-hydroxyphenylpyruvate dioxygenase, a D1 protein of photosystem II, a ribulose-bisphosphate carboxylase, a 5-enolpyruvylshikimate-3-phosphate synthase, a 3-deoxy-D-arabino-heptulosonate 7-phosphate synthase, an acetyl-coenzyme A carboxylase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

7. The method of any one of embodiments 1-6, wherein the essential gene encodes a regulatory subunit of an acetolactate synthase, a ketol-acid reductoisomerase, a dihydroxy-acid dehydratase, a gamma subunit of an ATP synthase, a 3-deoxy-D-arabino-heptulosonate 7-phosphate synthase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

8. The method of any one of embodiments 1-7, wherein the essential gene encodes a polypeptide having at least 80%, at least 90%, at least 95%, at least 98%, at least 99%, or 100% sequence identity to at least one of SEQ ID NOs: 35-68, 102-134, 168-200, 240-278, 317-354.

9. The method of any one of embodiments 1-8, wherein the weed is growing in a field of crop plants.

10. The method of embodiment 9, wherein the crop plant is corn, soybean, or wheat.

11. The method of embodiment 9 or embodiment 10, wherein the antisense oligonucleotide is not complementary to a transcript of an essential gene of the crop plant, optionally wherein the antisense oligonucleotide is not complementary to a transcript of any gene of the crop plant.

12. The method of any one of embodiments 1-11, wherein the weed is a monocot or a dicot.

13. The method of any one of embodiments 1-12, wherein the weed is Amaranthus palmeri, Lolium rigidum, Bassia scoparia, or Alopecurus myosuroides.

14. The method of any one of embodiments 1-13, wherein the weed is herbicide resistant.

15. The method of any one of embodiments 1-14, wherein the composition comprises two or more antisense oligonucleotides, optionally wherein the composition comprises from 2, 3, 4, 5, 6, 7, 8, 9, or 10 to 15, 20, 25, 30, 50, or 100 antisense oligonucleotides, optionally wherein the composition comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 antisense oligonucleotides.

16. The method of embodiment 15, wherein the antisense oligonucleotides target different segments of a transcript of the same gene.

17. The method of embodiment 15 or embodiment 16, wherein the antisense oligonucleotides target transcripts of multiple genes.

18. The method of any one of embodiments 1-17, wherein the composition further comprises a nanoparticle.

19. The method of embodiment 18, wherein the nanoparticle comprises a carbon dot.

20. The method of any one of embodiments 1-19, wherein the composition further comprises a carrier, a surfactant, or an herbicide.

21. The method of any one of embodiments 1-20, wherein the composition is in the form of a liquid, a powder, a granule, a paste, a pellet, or a gel.

22. The method of any one of embodiments 1-21, wherein the composition is provided as a foliar application, an in-furrow application, or a soil drench application.

23. A method of inducing gene silencing in a plant, the method comprising: providing to the plant a composition comprising a nanoparticle and an antisense oligonucleotide comprising at least one FANA-modified nucleotide, wherein the antisense oligonucleotide comprises a sequence complementary to at least 18, at least 19, at least 20, at least 21, at least 22, at least 23, at least 24, or at least 25 contiguous nucleotides of a transcript of a target gene of the plant.

24. The method of embodiment 23, wherein the antisense oligonucleotide comprises or consists of a sequence complementary to at least 18, at least 19, at least 20, at least 21, at least 22, at least 23, at least 24, or at least 25 contiguous nucleotides of at least one of SEQ ID NOs: 1-34, 69-101, 135-167, 201-239.

25. The method of embodiment 23 or embodiment 24, wherein the antisense oligonucleotide comprises or consists of a sequence complementary to 21 contiguous nucleotides of at least one of SEQ ID NOs: 1-34, 69-101, 135-167, 201-239.

26. The method of any one of embodiments 23-25, wherein the antisense oligonucleotide comprises or consists of the sequence of at least one of SEQ ID NOs: 533-567.

27. The method of any one of embodiments 23-26, wherein the target gene encodes a catalytic subunit of an acetolactate synthase, a regulatory subunit of an acetolactate synthase, a ketol-acid reductoisomerase, a dihydroxy-acid dehydratase, a gamma subunit of an ATP synthase, a protoporphyrinogen oxidase, a glutamine synthase, a phytoene desaturase, a 4-hydroxyphenylpyruvate dioxygenase, a D1 protein of photosystem II, a ribulose-bisphosphate carboxylase, a 5-enolpyruvylshikimate-3-phosphate synthase, a 3-deoxy-D-arabino-heptulosonate 7-phosphate synthase, an acetyl-coenzyme A carboxylase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

28. The method of any one of embodiments 23-27, wherein the target gene encodes a regulatory subunit of an acetolactate synthase, a ketol-acid reductoisomerase, a dihydroxy-acid dehydratase, a gamma subunit of an ATP synthase, a 3-deoxy-D-arabino-heptulosonate 7-phosphate synthase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

29. The method of any one of embodiments 23-28, wherein the target gene is an essential gene for maintaining the growth or life of the plant.

30. The method of any one of embodiments 23-29, wherein the target gene encodes a polypeptide that confers herbicide resistance to the plant.

31. The method of any one of embodiments 23-30, wherein the essential gene encodes a polypeptide having at least 80%, at least 90%, at least 95%, at least 98%, at least 99%, or 100% sequence identity to at least one of SEQ ID NOs: 35-68, 102-134, 168-200, 240-278, 317-354.

32. The method of any one of embodiments 23-31, wherein the nanoparticle comprises a carbon dot.

33. The method of any one of embodiments 23-31, wherein the composition comprises two or more antisense oligonucleotides, optionally wherein the composition comprises from 2, 3, 4, 5, 6, 7, 8, 9, or 10 to 15, 20, 25, 30, 50, or 100 antisense oligonucleotides, optionally wherein the composition comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 antisense oligonucleotides.

34. The method of embodiment 33, wherein the antisense oligonucleotides target different segments of a transcript of the same gene.

35. The method of embodiment 33 or embodiment 34, wherein the antisense oligonucleotides target transcripts of multiple genes.

36. An antisense oligonucleotide comprising at least one FANA-modified nucleotide, wherein the antisense oligonucleotide comprises a sequence complementary to at least 18, at least 19, at least 20, at least 21, at least 22, at least 23, at least 24, or at least 25 contiguous nucleotides of a transcript of an essential gene of a plant.

37. The antisense oligonucleotide of embodiment 36, wherein the antisense oligonucleotide comprises or consists of a sequence complementary to at least 18, at least 19, at least 20, at least 21, at least 22, at least 23, at least 24 or at least 25 contiguous nucleotides of at least one of SEQ ID NOs: 1-34, 69-101, 135-167, 201-239.

38. The antisense oligonucleotide of embodiment 36 or embodiment 37, wherein the antisense oligonucleotide comprises or consists of a sequence complementary to 21 contiguous nucleotides of at least one of SEQ ID NOs: 1-34, 69-101, 135-167, 201-239.

39. The antisense oligonucleotide of any one of embodiments 36-38, wherein the antisense oligonucleotide comprises or consists of the sequence of at least one of SEQ ID NOs: 533-567.

40. The antisense oligonucleotide of any one of embodiments 36-39, wherein the essential gene encodes a catalytic subunit of an acetolactate synthase, a regulatory subunit of an acetolactate synthase, a ketol-acid reductoisomerase, a dihydroxy-acid dehydratase, a subunit of a chloroplast ATP synthase, a protoporphyrinogen oxidase, a glutamine synthase, a phytoene desaturase, a 4-hydroxyphenylpyruvate dioxygenase, a D1 protein of photosystem II, a ribulose-bisphosphate carboxylase, a 5-enolpyruvylshikimate-3-phosphate synthase, a 3-deoxy-D-arabino-heptulosonate 7-phosphate synthase, an acetyl-coenzyme A carboxylase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

41. The antisense oligonucleotide of any one of embodiments 36-40, wherein the essential gene encodes a regulatory subunit of an acetolactate synthase, a ketol-acid reductoisomerase, a dihydroxy-acid dehydratase, a subunit of a chloroplast ATP synthase, a 3-deoxy-D-arabino-heptulosonate 7-phosphate synthase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

42. The antisense oligonucleotide of any one of embodiments 36-41, wherein the essential gene encodes a polypeptide having at least 80%, at least 90%, at least 95%, at least 98%, at least 99%, or 100% sequence identity to at least one of SEQ ID NOs: 35-68, 102-134, 168-200, 240-278, 317-354.

43. A composition comprising the antisense oligonucleotide of any one of embodiments 36-42.

44. The composition of embodiment 43, wherein the composition comprises two or more antisense oligonucleotides, optionally wherein the composition comprises from 2, 3, 4, 5, 6, 7, 8, 9, or 10 to 15, 20, 25, 30, 50, or 100 antisense oligonucleotides, optionally wherein the composition comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 antisense oligonucleotides.

45. The composition of embodiment 43 or embodiment 44, wherein the composition further comprises a nanoparticle.

46. The composition of any one of embodiments 43-45, wherein the nanoparticle comprises a carbon dot.

47. The composition of any one of embodiments 43-46, wherein the composition further comprises a carrier, a surfactant, or an herbicide.

48. The composition of any one of embodiments 43-47, wherein the composition is in the form of a liquid, a powder, a granule, a paste, a pellet, or a gel.

49. A plant or plant cell comprising the antisense oligonucleotide of any one of embodiments 27-42 or the composition of any one of embodiments 43-48.

50. A plant seed coated with a composition comprising the antisense oligonucleotide of any one of embodiments 27-42 or the composition of any one of embodiments 43-48.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

### Example 1: FANA Antisense Oligonucleotides Enter the Plant from Foliar Application

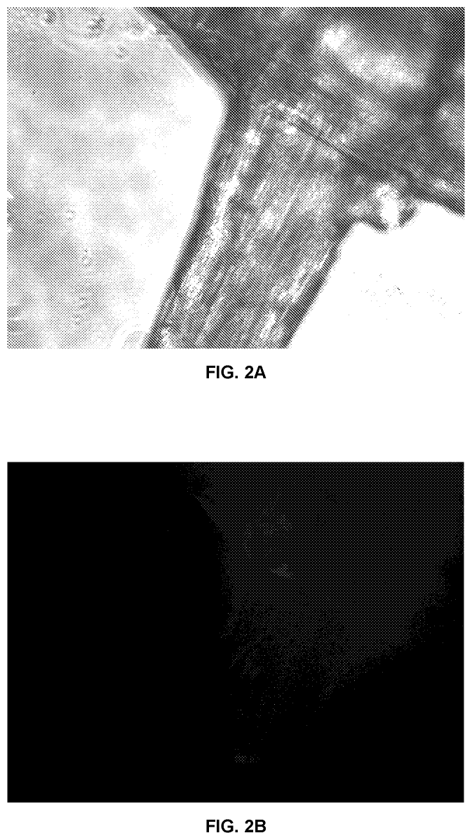

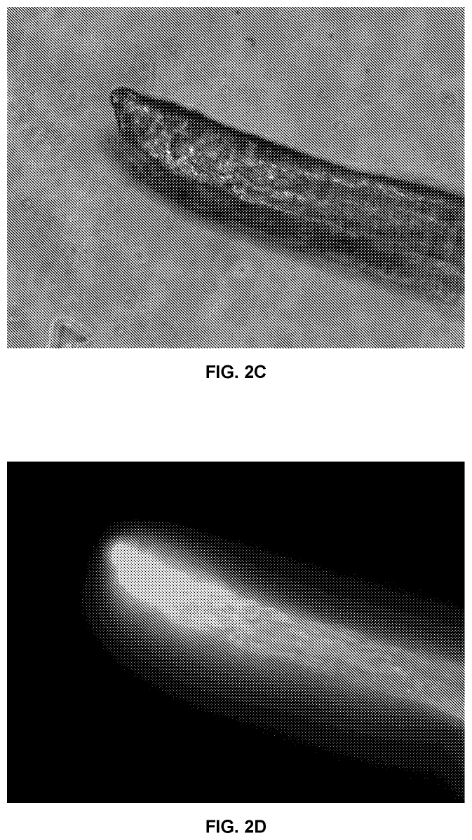

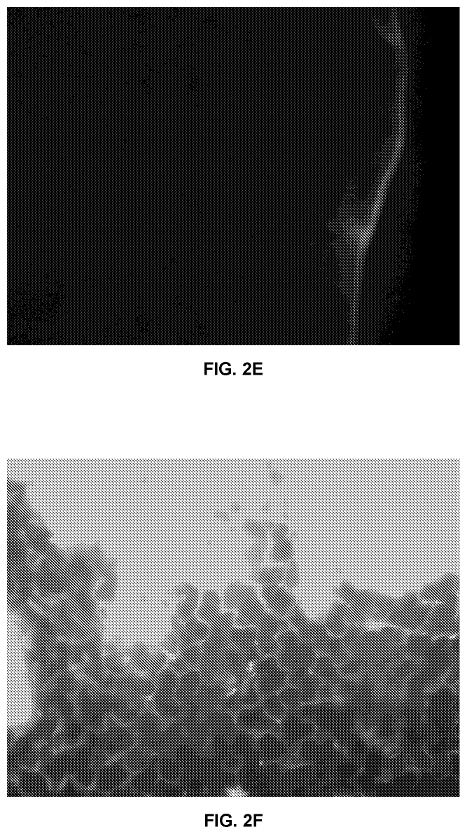

FANA antisense oligonucleotides enter the cytoplasm of plant cells when applied to the leaf without the use of nanoparticles. A fluorescent FAM-labeled FANA antisense oligonucleotide showed uptake into both plant roots and leaves (FIG. 2). Palmer amaranth seedlings were germinated on 0.7% agarose containing 20 μM fluorescent FAM-labeled FANA antisense oligonucleotide. Roots were washed at 3 days after germination and images collected. Palmer amaranth leaves were treated with a 10 μL of solution as 10 by 1 μL droplets containing 9 nmol (900 μM concentration) fluorescent FAM-labeled FANA antisense oligonucleotide along with 0.25% non-ionic surfactant, allowed to absorb the solution for 24 hours, and then leaves were washed and imaged at 20× magnification.

### Example 2: FANA Antisense Oligonucleotides Reduce mRNA Abundance

FANA antisense oligonucleotides targeting the mRNA of EPSPS, ALS, and HPPD genes in Palmer amaranth showed a reduction in transcript abundance at 12 hours after treatment (Table 5). The treatment was a mixture of six different FANA antisense oligonucleotides targeting ALS, EPSPS, and HPPD genes. The FANA antisense oligonucleotides used in this experiment were ALS_SEQ1, ALS_SEQ7, EPSPS_SEQ3, EPSPS_SEQ4, HPPD_SEQ1, and HPPD_SEQ4 with 72 nmol of each applied in 80 μL of application liquid containing 0.05% v/v nonionic surfactant (NIS). The sequences of the FANA antisense oligonucleotides are summarized in Table 6. The first emerging leaf was sampled at 12 hours after treatment, flash frozen in liquid nitrogen, and then used to extract mRNA for conversion to cDNA and measurement of transcript abundance in qRT-PCR with primers specific for each gene. Gene expression is shown as 2^{ΔCt} using GAPDH as a normalization gene.

TABLE 5

| | mRNA Transcript Abundance (Relative Expression) | | |
|---|---|---|---|
| Treatment | ALS | EPSPS | HPPD |
| Surfactant only | 0.8 | 0.76 | 0.56 |
| (NIS at 0.05% v/v) | | | |
| Scramble | 1.54 | 0.5 | 1.28 |
| FANA ASO mixture | 0.01 | 0.01 | 0.05 |

TABLE 6

| Gene | | |
|---|---|---|
| Target | Name | Antisense Oligonucleotide Sequence |
| ALS | ALS_SEQ1 | AAGAAGTGCATCAGCAAGACC (SEQ ID NO: 533) |
| | ALS_SEQ2 | TGGTATATCACATGCTTCAGC (SEQ ID NO: 534) |
| | ALS_SEQ3 | TGAGTCAAGAAGTGCATCAGC (SEQ ID NO: 535) |
| | ALS_SEQ4 | AGTAAGAGCTTGATGGATTTC (SEQ ID NO: 536) |
| | ALS_SEQ5 | ATCATCAGTACAAGGGAAAGC (SEQ ID NO: 537) |
| | ALS_SEQ6 | ACATTGTTTGAATTGCAGCCC (SEQ ID NO: 538) |
| | ALS_SEQ7 | AGTAAGAGCTTGATGGATTTC (SEQ ID NO: 539) |
| | ALS_SEQ8 | ACATGAGGTTGCTTATTCTTC (SEQ ID NO: 540) |
| EPSPS | EPSPS_SEQ1 | TTAGACCTGCTACCAGATCCC (SEQ ID NO: 541) |
| | EPSPS_SEQ2 | TTATCAACTATCTCAATCTCC (SEQ ID NO: 542) |
| | EPSPS_SEQ3 | AGAATATCATCACTATACAGC (SEQ ID NO: 543) |
| | EPSPS_SEQ4 | TAAAGTATGGTGCAATTGACC (SEQ ID NO: 544) |
| | EPSPS_SEQ5 | ACATGAACTTTGGAGAAATTC (SEQ ID NO: 545) |
| HPPD | HPPD_SEQ1 | ACACTCCTTCATCTGCTCCTC (SEQ ID NO: 546) |
| | HPPD_SEQ2 | TTCATCTTTCATCATGCAACC (SEQ ID NO: 547) |
| | HPPD_SEQ3 | AGCAATTGCAGGAGCCAACTC (SEQ ID NO: 548) |
| | HPPD_SEQ4 | TCGTCCCAACATCTTCAGCTG (SEQ ID NO: 549) |
| | HPPD_SEQ5 | TAGGAGAAACACAGGGAATAG (SEQ ID NO: 550) |
| ATPC1 | ATPC1-1 | TCTTGAGCTCGTCGGACTTTC (SEQ ID NO: 551) |
| | ATPC1-2 | TCAGAATCGTGTTGTTGAACC (SEQ ID NO: 552) |
| | ATPC1-3 | TACCTATCGACAGGAATTGCC (SEQ ID NO: 553) |
| | ATPC1-4 | TGTTGTCAGACGGAATAGCTC (SEQ ID NO: 554) |
| | ATPC1-5 | TGACTGTTTAGATATAATGGC (SEQ ID NO: 555) |
| PDS | PDS-1 | ATACTTTGCCGTGGATAGACC (SEQ ID NO: 556) |
| | PDS-2 | ATAGAGTGCTCCTTCCATTGC (SEQ ID NO: 557) |
| | PDS-3 | TTCAGAGCAATCAAGATGCAC (SEQ ID NO: 558) |
| | PDS-4 | TAGTGACTCAATGTGCTTAAC (SEQ ID NO: 559) |
| | PDS-5 | TCAGTGTCGCTTCGTGAAATC (SEQ ID NO: 560) |
| | PDS-6 | TCTATTGGTGATCTTTGCAGC (SEQ ID NO: 561) |
| | AMAPA_PDS_1 | TTGATGTGGATTGAGTAGCAC (SEQ ID NO: 562) |
| | AMAPA_PDS_2 | ATACTTTGCCGTGGATAAACC (SEQ ID NO: 563) |
| | AMAPA_PDS_3 | ATCAGTTACACGATCGGGTAC (SEQ ID NO: 564) |
| | AMAPA_PDS_4 | TAGTAACAGCTTCAAGATGTC (SEQ ID NO: 565) |
| | AMAPA_PDS_5 | TCTATTGGTGATCTTTGCAGC (SEQ ID NO: 566) |
| | AMAPA_PDS_6 | TCAGTGTCGCTTCGTGAAATC (SEQ ID NO: 567) |

### Example 3: FANA Antisense Oligonucleotides Cause Growth Inhibition in Palmer Amaranth

FANA antisense oligonucleotides inhibited growth of Palmer amaranth seedlings in agarose media. Four out of 12 tested gene-specific FANA antisense oligonucleotides reduced seedling growth with no effect from a scramble FANA antisense oligonucleotide (Table 7). FANA antisense oligonucleotides targeting mRNA transcripts of acetolactate synthase (ALS), 5-enolpyruvylshikimate-3-phosphate synthase (EPSPS), and 4-hydroxyphenylpyruvate dioxygenase (HPPD) were at concentrations ranging from 20 μM to 500 μM in the agarose solution. Ten seeds were placed on 0.7% agarose and evaluated 6 days after placing on the agarose. Seedlings with green cotyledons were indicative of a healthy plant. Seedlings were also rated scale from 0 (no visible effect or damage) to 10 (maximum damage). FANA antisense oligonucleotides with 0% seedlings with green cotyledons and/or a rating of 7 or higher were considered to have lethal activity on germinating seedlings. Scramble FANA antisense oligonucleotide negative control showed no reduction in green cotyledon growth or increase in damage rating. Seedlings in untreated and scramble treatments had extensive root hair growth, while no root hair growth occurred in highlighted treatments. Results of additional independent experiments are shown in Tables 8-10.

TABLE 7

| | | | Number | | |
|---|---|---|---|---|---|
| | Concentration | Number Total | Seedlings w/ | % Green | |
| Treatment | (μM) | Seedlings | Green Cotyledons | Cotyledons | Rating |
| Untreated 1 | — | 10 | 8 | 80 | 2 |
| Untreated 2 | — | 10 | 9 | 90 | 2 |
| Untreated 3 | — | 10 | 7 | 70 | 3 |
| Scramble | 200 | 10 | 10 | 100 | 1 |
| ALS SEQ1 | 20 | 9 | 8 | 89 | 2 |
| ALS SEQ1 | 200 | 10 | 0 | 0 | 6 |
| ALS SEQ3 | 20 | 10 | 9 | 90 | 0 |
| ALS SEQ3 | 200 | 7 | 4 | 57 | 6 |
| EPSPS SEQ1 | 20 | 10 | 9 | 90 | 1 |
| EPSPS SEQ1 | 200 | 9 | 6 | 67 | 6 |
| EPSPS SEQ2 | 20 | 9 | 9 | 100 | 1 |
| EPSPS SEQ2 | 200 | 9 | 1 | 11 | 7 |
| HPPD SEQ4 | 20 | 10 | 10 | 100 | 2 |
| HPPD SEQ4 | 500 | 8 | 1 | 13 | 7 |
| HPPD SEQ1 | 20 | 10 | 10 | 100 | 2 |
| HPPD SEQ1 | 500 | 8 | 0 | 0 | 8 |

TABLE 8

| | | | Number | | |
|---|---|---|---|---|---|
| | Concentration | Number Total | Seedlings w/ | % Green | |
| Treatment | (μM) | Seedlings | Green Cotyledons | Cotyledons | Rating |
| Untreated 1 | — | 9 | 8 | 89 | 2 |
| Untreated 2 | — | 10 | 8 | 80 | 3 |
| Untreated 3 | — | 8 | 5 | 63 | 1 |
| ALS SEQ2 | 250 | 9 | 4 | 44 | 4 |
| ALS SEQ8 | 250 | 10 | 6 | 60 | 4 |
| EPSPS SEQ4 | 250 | 9 | 0 | 0 | 8 |
| EPSPS SEQ5 | 250 | 9 | 5 | 56 | 6 |
| HPPD SEQ2 | 250 | 8 | 4 | 50 | 5 |

TABLE 9

| | | | Number | | |
|---|---|---|---|---|---|
| | Concentration | Number Total | Seedlings w/ | % Green | |
| Treatment | (μM) | Seedlings | Green Cotyledons | Cotyledons | Rating |
| Untreated 1 | — | 8 | 8 | 100 | 1 |
| Untreated 2 | — | 10 | 9 | 90 | 2 |
| ALS SEQ4 | 250 | 9 | 4 | 44 | 4 |
| ALS SEQ5 | 250 | 9 | 2 | 22 | 6 |
| ALS SEQ6 | 250 | 10 | 2 | 20 | 7 |
| ALS SEQ7 | 250 | 9 | 1 | 11 | 7 |
| EPSPS SEQ3 | 200 | 7 | 0 | 0 | 8 |

TABLE 10

| | | Number | Number | | |
|---|---|---|---|---|---|
| | Concentration | Total | Seedlings w/ | % Green | |
| Treatment | (μM) | Seedlings | Green Cotyledons | Cotyledons | Rating |
| Untreated 1 | — | 10 | 8 | 80 | 1 |
| Untreated 2 | — | 9 | 9 | 100 | 1 |
| ALS1 | 150 | 9 | 1 | 11 | 7 |
| HPPD1 | 100 | 10 | 2 | 20 | 6 |
| EPSPS3 | 100 | 9 | 4 | 44 | 5 |
| ALS7 | 100 | 9 | 6 | 67 | 3 |
| ALS7 + HPPD1 | 100 each | 9 | 2 | 22 | 5 |
| ALS7 + EPSPS3 | 100 each | 7 | 1 | 14 | 7 |
| HPPD1 + EPSPS3 | 100 each | 7 | 0 | 0 | 8 |
| ALS7 + HPPD1 + EPSPS3 | 100 each | 9 | 0 | 0 | 9 |

### Example 4: FANA Antisense Oligonucleotides Cause Herbicidal Symptoms in Palmer Amaranth

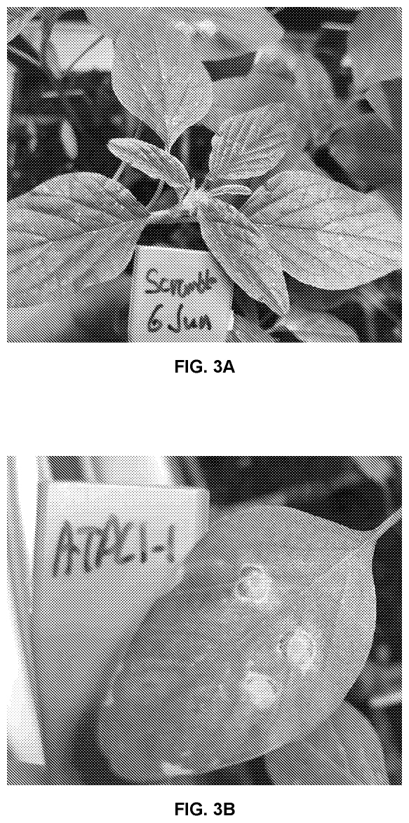

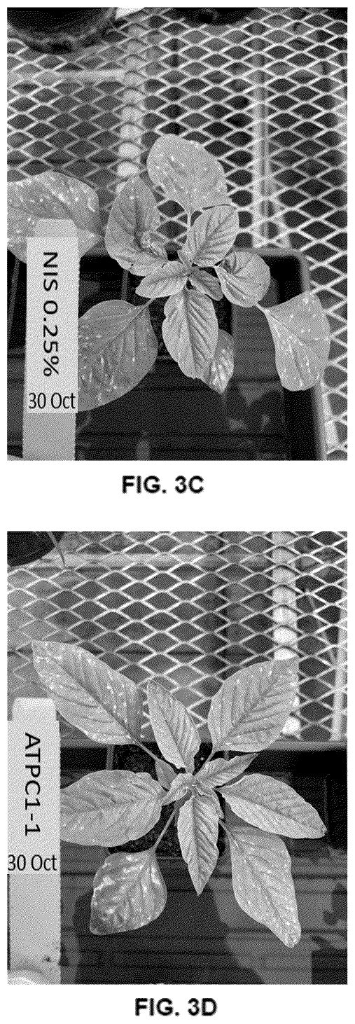

A FANA antisense oligonucleotide targeting ATPC1 caused herbicidal symptoms such as leaf necrosis when applied using syringe injection but not foliar spray (FIG. 3).

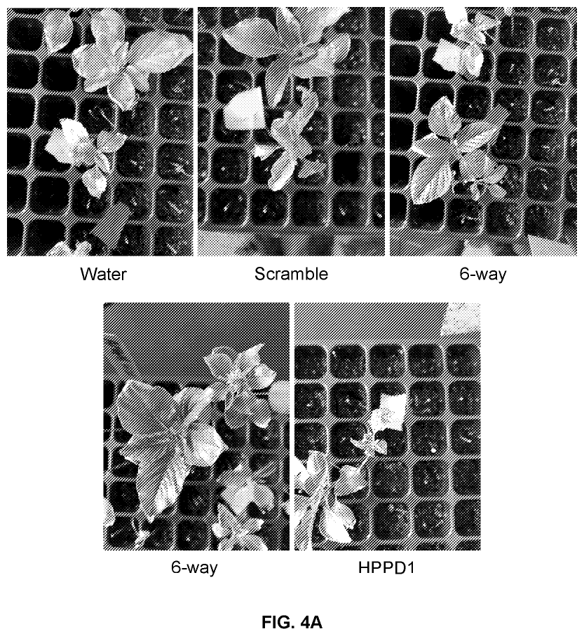

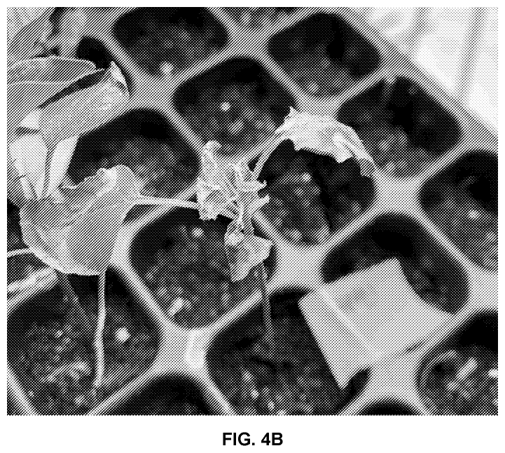

A lethal effect on Palmer amaranth when applied as syringe injection to the underside of plant leaves was observed with a FANA antisense oligonucleotide targeting the HPPD gene (HPPD_SEQ1) (FIG. 4A-B). Treatments consisted of 500 μL water containing water only; water plus 20 nmol scramble FANA antisense oligonucleotide; water plus 120 nmol total FANA antisense oligonucleotide consisting of two FANA antisense oligonucleotide sequences for each of three plant genes (20 nmol each FANA antisense oligonucleotide consisting of ALS_SEQ1, ALS_SEQ7, EPSPS_SEQ3, EPSPS_SEQ4, HPPD_SEQ1, and HPPD_SEQ2); and 720 nmol of FANA ASO HPPD_SEQ1. Photos were taken 18 days after treatment. The plant treated with FANA antisense oligonucleotide targeting HPPD showed typical symptoms of HPPD inhibition, including bleaching and chlorosis, followed by death.

FANA antisense oligonucleotides targeting either PDS (PDS-1 or PDS-2) or ATPC1 (ATPC1-1 or ATPC1-2) were applied by syringe injection to the underside of plant leaves. Treatments consisted of 500 μL water containing 160 nmol of the FANA antisense oligonucleotides (320 μM solution). Targeting either PDS or ATPC1 caused a phenotype consistent with inhibition of these genes at injection sites.

### Example 5: Carbon Dot Nanoparticles Improve FANA Antisense Oligonucleotide Uptake Across the Leaf Cuticle and into the Plant Nucleus

In this example, experiments were performed to evaluate whether carbon dots improve the uptake of FANA antisense oligonucleotides into plants.

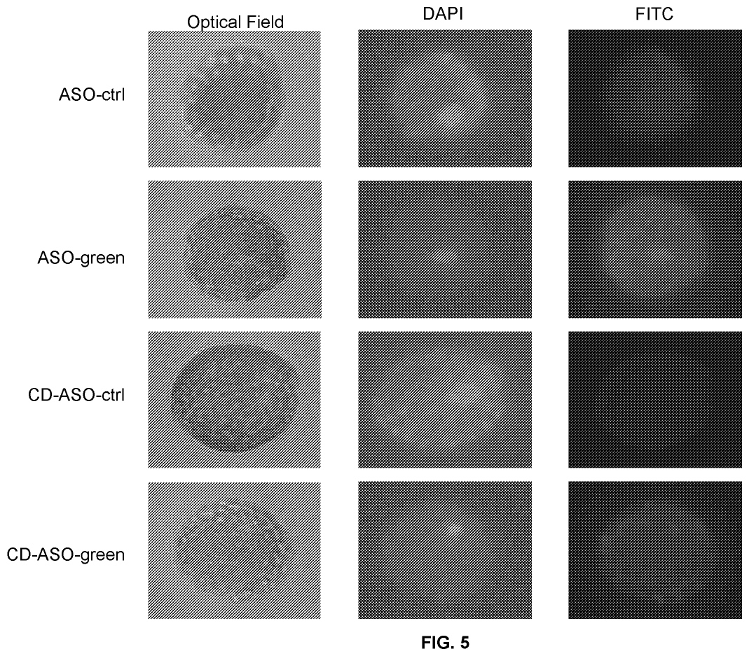

Protoplasts were isolated from Arabidopsis and treated with a fluorescent FANA antisense oligonucleotide or carbon dots with the FANA antisense oligonucleotide (CD-ASO). The results confirmed that the FANA antisense oligonucleotide reached the nucleus (FIG. 5).

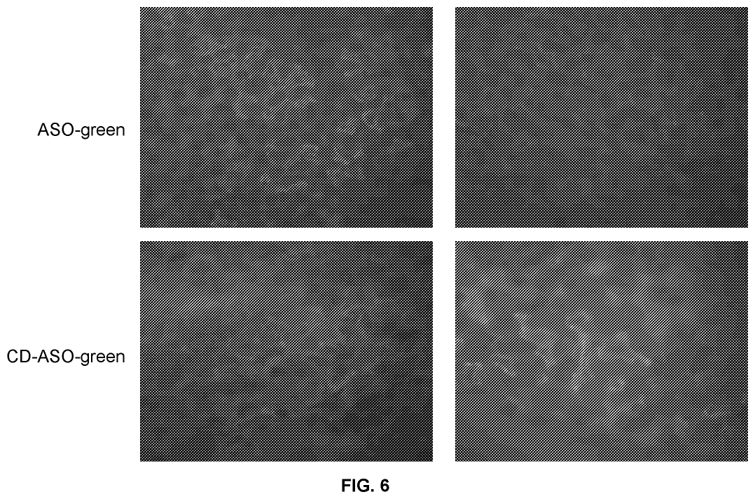

Arabidopsis leaves were infiltrated with a fluorescent FANA antisense oligonucleotide or carbon dots with the FANA antisense oligonucleotide. The carbon dot treated areas showed more cytoplasm uptake of the fluorescent FANA antisense oligonucleotide (FIG. 6).

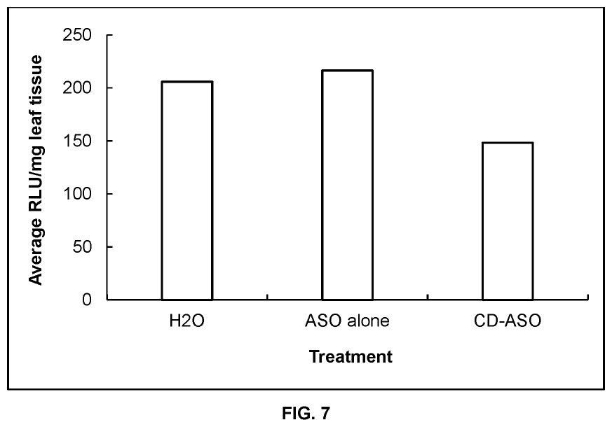

A luciferase assay was performed on leaves treated with a FANA antisense oligonucleotide or carbon dots with the FANA antisense oligonucleotide. Plant leaves were dotted with 20 μL of treatment solution per leaf and left for 7 days. The luciferase assay was performed on two individual leaves per treatment. The results indicated that carbon dots may improve FANA antisense oligonucleotide uptake into the plant (FIG. 7).

Small Palmer amaranth plants were sprayed with FANA antisense oligonucleotides targeting ATPC1 (ATPC1_1; SEQ ID NO: 551), HPPD (HPPD_1; SEQ ID NO: 546), ALS (ALS_1; SEQ ID NO: 533), or EPSPS (EPSPS_4: SEQ ID NO: 544) both with and without nanoparticles (carbon dots and single-walled carbon nanotubes). The treatment solution consisted of 50 nmol of the FANA antisense oligonucleotide, 5-10 mg of nanoparticles, and 0.05% NIS solution (Silwet L-77). Plants treated with the FANA antisense oligonucleotide targeting ATPC1 showed a yellowing and chlorosis phenotype starting at 1 day after treatment through 7 days after treatment (both with and without nanoparticles). One replicate of the FANA antisense oligonucleotide treatment with nanoparticles targeting ATPC1 showed gene silencing at 12 hours after treatment based on qPCR analysis.

### Example 6: Design of FANA Antisense Oligonucleotides to Selectively Target mRNA of Weed Genes

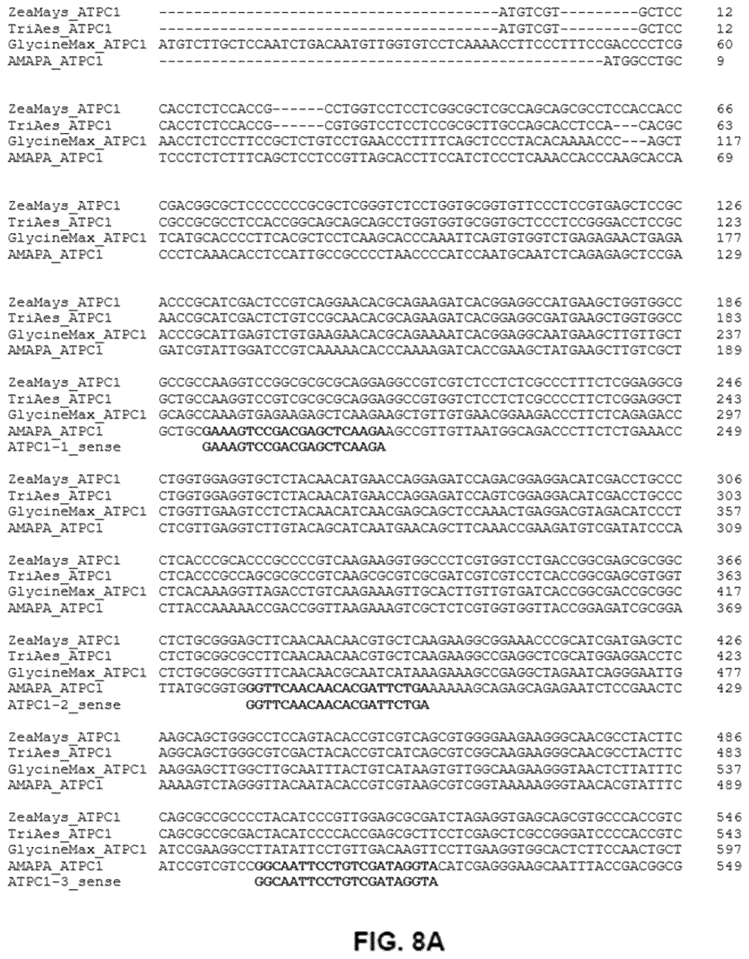

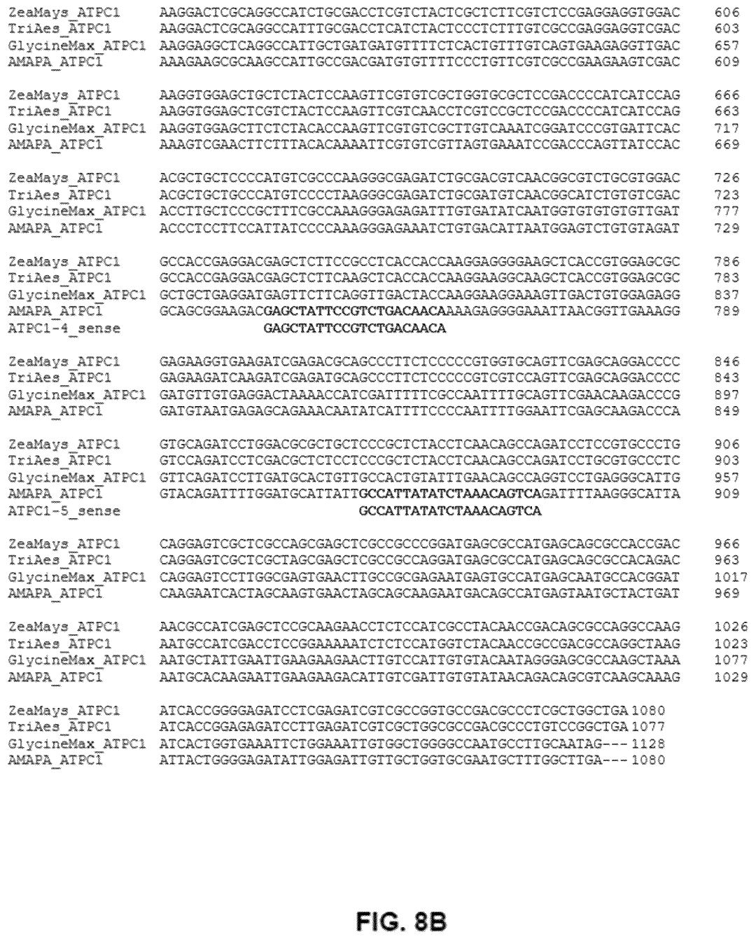

FANA antisense oligonucleotides can be designed to selectively target mRNA of weed genes and not target the corresponding mRNA of the gene in crops because the sequence of the FANA antisense oligonucleotide matches the weed and has at least one or more base pair mismatches with the crop sequence. As an example, FIG. 8A-B shows a multiple sequence alignment of the ATPC1 sequences for Palmer amaranth (AMAPA, Amaranthus palmeri), maize (Zea mays), soybean (Glycine max), wheat (Triticum aestivum), and five FANA antisense oligonucleotide sequences (shown in sense orientation) specifically targeting ATPC1 in Palmer amaranth.

What is claimed is:

1. A method of controlling weeds, the method comprising:
providing to the weed a composition comprising an antisense oligonucleotide comprising at least one FANA-modified nucleotide, wherein the antisense oligonucleotide targets a transcript of an essential gene of the weed.

2. The method of claim 1, wherein the antisense oligonucleotide comprises a sequence complementary to at least 18, at least 19, at least 20, at least 21, at least 22, at least 23, at least 24, or at least 25 contiguous nucleotides of the transcript.

3. The method of claim 1, wherein the antisense oligonucleotide comprises a sequence complementary to at least 18, at least 19, at least 20, at least 21, at least 22, at least 23, at least 24, or at least 25 contiguous nucleotides of at least one of SEQ ID NOs: 1-34, 69-101, 135-167, 201-239.

4. The method of claim 1, wherein the antisense oligonucleotide consists of a sequence complementary to 21 contiguous nucleotides of at least one of SEQ ID NOs: 1-34, 69-101, 135-167, 201-239.

5. The method of claim 1, wherein the antisense oligonucleotide comprises the sequence of at least one of SEQ ID NOs: 533-567.

6. The method of claim 1, wherein the essential gene encodes a catalytic subunit of an acetolactate synthase, a regulatory subunit of an acetolactate synthase, a ketol-acid reductoisomerase, a dihydroxy-acid dehydratase, a gamma subunit of an ATP synthase, a protoporphyrinogen oxidase, a glutamine synthase, a phytoene desaturase, a 4-hydroxyphenylpyruvate dioxygenase, a D1 protein of photosystem II, a ribulose-bisphosphate carboxylase, a 5-enolpyruvylshikimate-3-phosphate synthase, a 3-deoxy-D-arabino-heptulosonate 7-phosphate synthase, an acetyl-coenzyme A carboxylase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

7. The method of claim 1, wherein the essential gene encodes a regulatory subunit of an acetolactate synthase, a ketol-acid reductoisomerase, a dihydroxy-acid dehydratase, a gamma subunit of an ATP synthase, a 3-deoxy-D-arabino-heptulosonate 7-phosphate synthase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

8. The method of claim 1, wherein the essential gene encodes a polypeptide having at least 80%, at least 90%, at least 95%, at least 98%, at least 99%, or 100% sequence identity to at least one of SEQ ID NOs: 35-68, 102-134, 168-200, 240-278, 316-352.

9. The method of claim 1, wherein the weed is growing in a field of crop plants.

10. The method of claim 9, wherein the crop plant is corn, soybean, or wheat.

11. The method of claim 9, wherein the antisense oligonucleotide is not complementary to a transcript of an essential gene of the crop plant.

12. The method of claim 1, wherein the weed is a monocot or a dicot.

13. The method of claim 1, wherein the weed is Amaranthus palmeri, Lolium rigidum, Bassia scoparia, or Alopecurus myosuroides.

14. The method of claim 1, wherein the weed is herbicide resistant.

15. The method of claim 1, wherein the composition comprises two or more antisense oligonucleotides.

16. The method of claim 15, wherein the antisense oligonucleotides target different segments of a transcript of the same gene.

17. The method of claim 15, wherein the antisense oligonucleotides target transcripts of multiple genes.

18. The method of claim 1, wherein the composition further comprises a nanoparticle.

19. The method of claim 18, wherein the nanoparticle comprises a carbon dot.

20. The method of claim 1, wherein the composition further comprises a carrier, a surfactant, or an herbicide.

21. The method of claim 1, wherein the composition is in the form of a liquid, a powder, a granule, a paste, a pellet, or a gel.

22. The method of claim 1, wherein the composition is provided as a foliar application, in-furrow application, or a soil drench application.

23. A method of inducing gene silencing in a plant, the method comprising:
providing to the plant a composition comprising a nanoparticle and an antisense oligonucleotide comprising at least one FANA-modified nucleotide, wherein the antisense oligonucleotide comprises a sequence complementary to at least 18, at least 19, at least 20, at least 21, at least 22, at least 23, at least 24, or at least 25 contiguous nucleotides of a transcript of a target gene of the plant.

24. The method of claim 23, wherein the nanoparticle comprises a carbon dot.

25. The method of claim 23, wherein the target gene is an essential gene for maintaining the growth or life of the plant.

26. The method of claim 23, wherein the target gene encodes a polypeptide that confers herbicide resistance to the plant.

27. An antisense oligonucleotide comprising at least one FANA-modified nucleotide, wherein the antisense oligonucleotide comprises a sequence complementary to at least 18, at least 19, at least 20, at least 21, at least 22, at least 23, at least 24, or at least 25 contiguous nucleotides of a transcript of an essential gene of a plant.

28. The antisense oligonucleotide of claim 27, wherein the antisense oligonucleotide comprises a sequence complementary to at least 18, at least 19, at least 20, at least 21, at least 22, at least 23, at least 24, or at least 25 contiguous nucleotides of at least one of SEQ ID NOs: 1-34, 69-101, 135-167, 201-239.

29. The antisense oligonucleotide of claim 27, wherein the antisense oligonucleotide consists of a sequence complementary to 21 contiguous nucleotides of at least one of SEQ ID NOs: 1-34, 69-101, 135-167, 201-239.

30. The antisense oligonucleotide of claim 27, wherein the antisense oligonucleotide comprises the sequence of at least one of SEQ ID NOs: 533-567.

31. The antisense oligonucleotide of claim 27, wherein the essential gene encodes a catalytic subunit of an acetolactate synthase, a regulatory subunit of an acetolactate synthase, a ketol-acid reductoisomerase, a dihydroxy-acid dehydratase, a subunit of a chloroplast ATP synthase, a protoporphyrinogen oxidase, a glutamine synthase, a phytoene desaturase, a 4-hydroxyphenylpyruvate dioxygenase, a D1 protein of photosystem II, a ribulose-bisphosphate carboxylase, a 5-enolpyruvylshikimate-3-phosphate synthase, a 3-deoxy-D-arabino-heptulosonate 7-phosphate synthase, an acetyl-coenzyme A carboxylase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

32. The antisense oligonucleotide of claim 27, wherein the essential gene encodes a regulatory subunit of an acetolactate synthase, a ketol-acid reductoisomerase, a dihydroxy-acid dehydratase, a subunit of a chloroplast ATP synthase, a 3-deoxy-D-arabino-heptulosonate 7-phosphate synthase, a fatty acyl-ACP thioesterase, a 3-ketoacyl-CoA synthase, a ceramide synthase, a mevalonate kinase, a dihydrodipicolinate synthase, an alpha-tubulin, a phosphoenolpyruvate carboxylase, or a pyruvate orthophosphate dikinase.

33. The antisense oligonucleotide of claim 27, wherein the essential gene encodes a polypeptide having at least 80%, at least 90%, at least 95%, at least 98%, at least 99%, or 100% sequence identity to at least one of SEQ ID NOs: 35-68, 102-134, 168-200, 240-278, 317-354.

34. An herbicidal composition comprising the antisense oligonucleotide of claim 27, and a carrier.

35. The herbicidal composition of claim 34, wherein the composition comprises two or more antisense oligonucleotides.

36. The herbicidal composition of claim 34, wherein the composition further comprises a nanoparticle.

37. The herbicidal composition of claim 34, wherein the nanoparticle comprises a carbon dot.

38. The herbicidal composition of claim 34, wherein the composition further comprises a surfactant or an herbicide.

39. The herbicidal composition of claim 34, wherein the composition is in the form of a liquid, a powder, a granule, a paste, a pellet, or a gel.

40. A plant or plant cell comprising the antisense oligonucleotide of claim 27.

41. A plant seed coated with a composition comprising the antisense oligonucleotide of claim 27.
